SEQUENCE LISTING (40 sequences)

SEQ ID NO: 1 - DNA, 53 bp, Artificial Sequence
  Primer petF3conA-Rlink
gtgactcagcggacatgcccggacatgccccaggtgcggttgctggcgcctat

SEQ ID NO: 2 - DNA, 53 bp, Artificial Sequence
  Primer petF4conA-Flink
gctgagtcacgggcatgtccgggcatgtccgatgcgtccggcgtagaggatcg

SEQ ID NO: 3 - DNA, 19 bp, Artificial Sequence
  Primer petF2
catcggtgatgtcggcgat

SEQ ID NO: 4 - DNA, 24 bp, Artificial Sequence
  Primer petR
cggatatagttcctcctttcagca

SEQ ID NO: 5 - DNA, 34 bp, Artificial Sequence
  Primer Hdm2-Nde1
cacaacatatgtgcaataccaacatgtctgtacc

SEQ ID NO: 6 - DNA, 55 bp, Artificial Sequence
  Primer Hdm2-HA-BamH1
gctctggatccttaagcgtaatctggaacatcgtatgggtaggggaaataagtta

SEQ ID NO: 7 - DNA, 40 bp, Artificial Sequence
  Primer INF-Hdm2-cmvF
cgaacctaaaaacaaatgtgcaataccaacatgtctgtac

SEQ ID NO: 8 - DNA, 33 bp, Artificial Sequence
  Primer INF-HA-cmvRcor
ttatagacaggtcaactaagcgtaatctggaac

SEQ ID NO: 9 - DNA, 31 bp, Artificial Sequence
  Primer mdm2-T16A-QC1
gatggtgctgtaaccgcctcacagattccag

SEQ ID NO: 10 - DNA, 31 bp, Artificial Sequence
  Primer mdm2-T16A-QC2
ctggaatctgtgaggcggttacagcaccatc

SEQ ID NO: 11 - DNA, 31 bp, Artificial Sequence
  Primer mdm2-P20L-QC1
ccacctcacagattctagcttcggaacaaga

SEQ ID NO: 12 - DNA, 31 bp, Artificial Sequence
  Primer mdm2-P20L-QC2
tcttgttccgaagctagaatctgtgaggtgg

SEQ ID NO: 13 - DNA, 31 bp, Artificial Sequence
  Primer mdm2-Q24R-QC1
ttccagcttcggaacgagagaccctggttag

SEQ ID NO: 14 - DNA, 31 bp, Artificial Sequence
  Primer mdm2-Q24R-QC2
ctaaccagggtctctcgttccgaagctggaa

SEQ ID NO: 15 - DNA, 34 bp, Artificial Sequence
  Primer HDMM62A-1
cttggccagtatattgcgactaaacgattatatg

SEQ ID NO: 16 - DNA, 34 bp, Artificial Sequence
  Primer HDMM62A-2
catataatcgtttagtcgcaatatactggccaag

SEQ ID NO: 17 - DNA, 31 bp, Artificial Sequence
  Primer mdm2-M62V-QC1
cttggccagtatattgtgactaaacgattat

SEQ ID NO: 18 - DNA, 31 bp, Artificial Sequence
  Primer mdm2-M62V-QC2
ataatcgtttagtcacaatatactggccaag

SEQ ID NO: 19 - DNA, 31 bp, Artificial Sequence
  Primer mdm2-V280A-QC1
tatatcaagttactgcgtatcaggcagggga

SEQ ID NO: 20 - DNA, 31 bp, Artificial Sequence
  Primer mdm2-V280A-QC2
tcccctgcctgatacgcagtaacttgatata

SEQ ID NO: 21 - DNA, 31 bp, Artificial Sequence
  Primer mdm2-G443D-QC1
gtgtgatttgtcaagatcgacctaaaaatgg

SEQ ID NO: 22 - DNA, 31 bp, Artificial Sequence
  Primer mdm2-G443D-QC2
ccatttttaggtcgatcttgacaaatcacac

SEQ ID NO: 23 - DNA, 17 bp, Artificial Sequence
  Primer 2CONART-F
ggcatgtccgctgagtc

SEQ ID NO: 24 - DNA, 22 bp, Artificial Sequence
  Primer WpetR1
taatttcgcgggatcgagatct

SEQ ID NO: 25 - DNA, 31 bp, Artificial Sequence; identical to SEQ ID NO: 11
  Primer HDM2-P20L-QC1

SEQ ID NO: 26 - DNA, 31 bp, Artificial Sequence; identical to SEQ ID NO: 12
  Primer HDM2-P20L-QC2

SEQ ID NO: 27 - DNA, 31 bp, Artificial Sequence; identical to SEQ ID NO: 13
  Primer HDM2-Q24R-QC1

SEQ ID NO: 28 - DNA, 31 bp, Artificial Sequence; identical to SEQ ID NO: 14
  Primer HDM2-Q24R-QC2

SEQ ID NO: 29 - DNA, 34 bp, Artificial Sequence; identical to SEQ ID NO: 15
  Primer HDM2-M62A-1

SEQ ID NO: 30 - DNA, 34 bp, Artificial Sequence; identical to SEQ ID NO: 16
  Primer HDM2-M62A-2

SEQ ID NO: 31 - DNA, 24 bp, Artificial Sequence
  Primer h_p21_Forward
gaggccgggatgagttgggaggag

SEQ ID NO: 32 - DNA, 24 bp, Artificial Sequence
  Primer h_p21_Reverse
cagccggcgtttggagtggtagaa

SEQ ID NO: 33 - DNA, 24 bp, Artificial Sequence
  Primer h_p53_forward
cccctcctggcccctgtcatcttc

SEQ ID NO: 34 - DNA, 24 bp, Artificial Sequence
  Primer h_p53_Reverse
gcagcgcctcacaacctccgtcat

SEQ ID NO: 35 - DNA, 25 bp, Artificial Sequence
  Primer h_b-actin_forward
tcacccacactgtgcccatctacga

SEQ ID NO: 36 - DNA, 25 bp, Artificial Sequence
  Primer h_b-actin_reverse
cagcggaaccgctcattgccaatgg

SEQ ID NO: 37 - DNA, 21 bp, Artificial Sequence
  Primer h_Gadd45alpha_forward
gagagcagaagaccgaaagga

SEQ ID NO: 38 - DNA, 19 bp, Artificial Sequence
  Primer h_Gadd45alpha_reverse
cagtgatcgtgcgctgact

SEQ ID NO: 39 - DNA, 20 bp, Artificial Sequence
  Primer h_14-3-3sigma_forward
actacgagatcgccaacagc

SEQ ID NO: 40 - DNA, 20 bp, Artificial Sequence
  Primer h-14-3-3sigma_reverse
cagtgtcaggttgtctcgca

(19) United States
(12) Patent Application Publication
(10) Pub. No.: US 2015/0276755 A1
(43) Pub. Date: Oct. 1, 2015

(54) METHODS FOR DETERMINING RESISTANCE AGAINST MOLECULES TARGETING PROTEINS

(71) Applicant: AGENCY FOR SCIENCE, TECHNOLOGY AND RESEARCH, Singapore (SG)

(72) Inventors: Farid Ghadessy, Singapore (SG); Chandra Shekhar Verma, Singapore (SG); David Philip Lane, Singapore (SG); Jia Wei Siau, Singapore (SG); Thomas Leonard Joseph, Singapore (SG); Adelene Yen Ling Sim, Singapore (SG)

(21) Appl. No.: 14/438,469

(22) PCT Filed: Oct. 25, 2013
(86) PCT No.: PCT/SG2013/000460

§ 371 Date: Apr. 24, 2015

(30) Foreign Application Priority Data
Oct. 25, 2012 (SG) 201207942-2

(51) Int. Cl. G01N 33/68; C12Q 1/68

(52) U.S. Cl. CPC G01N 33/6845; C12Q 1/6883; G01N 2333/4748; G01N 2333/9015; G01N 2500/02; G01N 2333/4704; G01N 2500/10; C12Q 2600/156

(57) ABSTRACT

The present disclosure provides a method of determining resistance of a biological molecule to inhibition of its interaction with a target molecule by an inhibitor of the biological molecule, the method comprising the steps of: a) co-compartmentalizing a gene encoding the biological molecule with the target molecule, or a gene encoding the biological molecule with a gene encoding the target molecule into an aqueous droplet disposed within a water-in-oil emulsion, and b) assaying for a complex comprising the biological molecule and the target molecule upon expression of the gene encoding the biological molecule and the gene encoding the target molecule, wherein detection of the complex in the presence of the inhibitor indicates that the biological molecule is resistant to inhibition of its interaction with the target molecule by the inhibitor. Also provided are mutated HDM2 ubiquitin ligase polypeptides exhibiting resistance to Nutlin inhibition of p53 binding.

### TECHNICAL FIELD

The present invention generally relates to methods for determining drug resistance. In particular, the present invention relates to methods for determining resistance to drug molecules that target protein-protein interactions.

### BACKGROUND

Drug resistance is a major therapeutic bottleneck that mandates a detailed understanding of any potential molecular escape mechanisms that may arise in patients. Many patients die as a result of the disease developing resistance to the drugs that are being used to treat them.

In order to counter this problem, it is important to be able to predict how readily diseases can become resistant to a particular drug. Knowledge of drug-specific resistance mechanisms, coupled with the ability to screen for these, will enable medical professionals to screen for these drug-resistant diseases during treatment and take relevant steps when they arise. Additionally, such knowledge before a drug goes into the clinic would facilitate the design of improved versions that would make it more difficult for the disease to adapt to.

Cancer is a major disease in the developed world. While significant improvements have been made in the efficacy of drugs used to treat cancer, the emergence of drug-resistance remains a significant problem in cancer treatment. Cancers are often able to adapt such that the drugs used to treat them are no longer effective. In order to address this problem, it is important to be able to predict how readily cancers can become resistant to a particular drug. Understanding drug-specific resistance mechanisms, together with the ability to screen for such resistance, may facilitate both rational therapeutic approaches and enable the development of next-generation drugs tailored to counteract the deleterious mutations in the target protein that confers the resistance.

The experimental approach to anticipating cancer drug resistance has most commonly involved the treatment of drug-sensitive cell lines, followed by analysis of resistant subpopulations [49]. Non-targeted in vitro mutagenesis of a cell line, followed by treatment and selection for resistance has also been described [50]. The analysis typically focuses on the cellular target and pathway that the drug acts on and can be extended to more high-throughput “omic” approaches such as expression profiling and whole exome sequencing. Whilst these analytical approaches have worked, there is the possibility of numerous events contributing to the resistance phenotype (e.g. gene mutation, gene deletion/duplication, promoter mutations, aberrant expression etc.), which may result in confounding analysis.

To circumvent this, target-based mutagenesis approaches have been adopted, wherein complementation by a mutated protein enables survival of an otherwise drug-sensitive cell line. These approaches, have accurately predicted drug resistance in several instances [51,52]. However, a major disadvantage of such approaches is the relatively small library of target protein variants that can be sampled due to inherent technical limitations arising from transformation efficiency (˜10⁶), and the possibility of off-target drug toxicity at higher doses that limits selection pressure. Furthermore, the viral transduction method used to stably introduce genes encoding mutant proteins introduces significant heterogeneity arising from the random nature of integration into the chromosome. This can impart significant bias due to variation in expression levels of the mutant proteins being screened.

Therefore, there is a need to provide a method for determining and predicting drug resistance that overcomes, or at least ameliorates, one or more of the disadvantages described above.

### SUMMARY

Disclosed herein is a method for determining and predicting drug resistance. In a first aspect, there is provided a method of determining resistance of a biological molecule to inhibition of its interaction with a target molecule by an inhibitor of the biological molecule, the method comprising the steps of:
a) co-compartmentalizing a gene encoding the biological molecule with the target molecule, or a gene encoding the biological molecule with a gene encoding the target molecule into an aqueous droplet disposed within a water-in-oil emulsion, and
b) assaying for a complex comprising the biological molecule and the target molecule upon expression of the gene encoding the biological molecule and the gene encoding the target molecule,
wherein detection of the complex in the presence of the inhibitor indicates that the biological molecule is resistant to inhibition of its interaction with the target molecule by the inhibitor, and
wherein non-detection of the complex in the presence of the inhibitor indicates that the biological molecule is not resistant to inhibition of its interaction with the target molecule by the inhibitor.

Advantageously, the disclosed method provides a completely cell-free methodology for determining resistance of a biological molecule to its inhibitor. Being completely cell-free, the disclosed method allows the use of stringent selection pressures for selecting biological molecules with exceptionally strong resistance phenotype, as well as with enhanced accuracy.

In one embodiment, the complex comprises the gene encoding the biological molecule. Advantageously, this enables selection and isolation of biological molecules having resistance phenotype, which can be used to facilitate design of new drugs or new versions of the drug to overcome the resistance.

In one embodiment, the emulsion comprises a plurality of the aqueous droplets. In one embodiment, each aqueous droplet comprises a single variant of the gene encoding the biological molecule. Advantageously, the provision of a plurality of the aqueous droplets each comprising a single variant of the gene encoding the biological molecule allows for high-throughput screening of a large repertoire of variants.

In a second aspect, there is provided a mutated HDM2 ubiquitin ligase polypeptide comprising at least one mutation selected from the group consisting of E69A, D225G, V241A, V280A, K344R, E390G, V426A, Q442R, M459T, Q24R, M62V, E124G, C461Y, T16A, P20L, L254F, N309T, G443D, H457R, L82P, and combinations thereof.

In a third aspect, there is provided a DNA molecule encoding the mutated HDM2 ubiquitin ligase polypeptide as defined above.

In a fourth aspect, there is provided a prognostic method for determining the receptiveness of a cancer patient to treatment with an anti-cancer drug capable of inhibiting the interaction of HDM2 ubiquitin ligase with p53 tumor suppressor protein, the method comprising the step of:
comparing a gene encoding the HDM2 ubiquitin ligase derived from a sample of the patient against a plurality of HDM2 ubiquitin ligase genes that have been determined to be resistant to the anti-cancer drug by the method according to the first aspect;
wherein identification of a match between the gene of the patient to at least one gene in the plurality of HDM2 ubiquitin ligase genes that have been determined to be resistant to the anti-cancer drug indicates that the cancer patient may not be receptive to treatment with the anti-cancer drug.

In a fifth aspect, there is provided a kit for use in a method of the first aspect, wherein the kit comprises means to co-compartmentalize the gene encoding the biological molecule with the target molecule or the gene encoding the target molecule into aqueous droplets disposed within a water-in-oil emulsion, and means to detect the complex comprising the biological molecule and the target molecule upon expression of the gene encoding the biological molecule and the gene encoding the target molecule.

In a sixth aspect, there is provided a method for selecting a variant form of a biological molecule that is resistant to inhibition of its interaction with a target molecule by an inhibitor of the biological molecule, comprising the steps of providing a plurality of randomly mutated genes encoding the biological molecule, and determining resistance of the biological molecule to inhibition of its interaction with the target molecule by the inhibitor using the method according to the first aspect.

In a seventh aspect, there is provided a kit for use in a method of the sixth aspect, comprising means for generating the plurality of randomly mutated genes encoding the biological molecule.

In an eighth aspect, there is provided a method of restoring the inhibitory activity of a drug on the interaction of a biological molecule with a target molecule, the method comprising the steps of:

i) identifying a variant of the biological molecule that is resistant to inhibition of its interaction with the target molecule by the drug, using the method of the first aspect; and

ii) modifying the drug to restore its inhibitory activity on the interaction of the biological molecule with the target molecule.

Advantageously, the method enables iterative improvement of the functionality and efficacy of a drug that is already in use or that is currently in development for clinical applications to reduce or avoid resistance.

### DEFINITIONS

Unless otherwise defined, the technical, scientific and medical terminology used herein has the same meaning as understood by those skilled in the art to which this invention belongs. However, for the purposes of establishing support for various terms that are used in the present application, the following technical comments, definitions and review are provided for reference. These are intended as general definitions and should in no way limit the scope of the present invention to those terms alone, but are put forth for a better understanding of the following description.

As used herein, the term “comprising” means “including”. Variations of the word “comprising”, such as “comprise” and “comprises,” have correspondingly varied meanings. Thus, for example, a composition “comprising” X may consist exclusively of X or may include one or more additional components.

As used herein, the term “about” as used in relation to a numerical value means, for example, +50% or +30% of the numerical value, preferably +20%, more preferably +10%, more preferably still +5%, and most preferably +1%. Where necessary, the word “about” may be omitted from the definition of the invention.

The term “or” means “and/or” unless explicitly indicated to refer to alternatives only or if the alternatives are mutually exclusive. The term “inhibition” as used herein refers to the act of decreasing, blocking, suppressing, or disrupting a particular activity or interaction such as the interaction between two molecules; or blocking, suppressing or disrupting a biological process, for example, an enzymatic reaction, gene expression, and the like.

The term “inhibitor” therefore refers to any molecule which has the ability to decrease, block, suppress, or disrupt a particular activity of another molecule, or the interaction between two other molecules.

The term “resistance to inhibition” is thus construed to mean the ability of a molecule to resist or withstand inhibition by an inhibitor, thereby maintaining its original activity or function, or its original interaction with other molecules in the presence of the inhibitor.

The term “wild-type” refers to a phenotype, genotype, or gene that predominates in a natural population of organisms or strain of organisms in contrast to that of natural or laboratory mutant forms.

The terms “mutant” and “mutation” include any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence.

The term “variant” may also be used to indicate a modified or altered form of a gene, DNA sequence, enzyme, cell, etc., i.e., any kind of mutant. For example, a mutant HDM2 ubiquitin ligase polypeptide comprising a L82P mutation is a variant form of the wild-type HDM2 ubiquitin ligase polypeptide. Typically, a “variant” will have substantially similar polypeptide or nucleic acid sequences as the “non-variant” (or wild-type) form. These variants may have at least 25%, 30%, 35%, 40%, 45%, 50%, 55%, 60%, 65%, 70%, 75%, 80%, 85%, 90%, 95%, 96%, 97%, 98% or 99% sequence identity to the “non-variant” polypeptide or nucleic acid. Variants may be made using, for example, the methods of protein engineering and site-directed mutagenesis as is well known in the art. For example, mutations may be introduced by chemical or physical mutagenic techniques, or using insertional mutation means such as transposons or T-DNA, and exogenous nucleic acid may be introduced by recombinant means employing, for example, chemical assisted cell permeation (using, for example, calcium, lithium, PEG), electroporation, microinjection, liposome-mediated transfection, microparticle bombardment (biolistics), virus infection, or any other appropriate means as are known in the art.

The term “analogue” refers to a chemical compound that is structurally similar to a parent compound or has chemical properties or pharmaceutical activity in common with the parent compound. Analogues include, but are not limited to, homologues, i.e., where the analogue differs from the parent compound by one or more carbon atoms in series; positional isomers; compounds that differ by interchange of one or more atoms by a different atom, for example, replacement of a carbon atom with an oxygen, sulfur, or nitrogen atom; and compounds that differ in the identity of one or more functional groups, for example, the parent compound differs from its analogue by the presence or absence of one or more suitable substituents. Suitable substituents include, but are not limited to, (C₁-C₈)alkyl; (C₁-C₈)alkenyl; (C₁-C₈)alkynyl; aryl; (C₂-C₅)heteroaryl; (C₁-C₆)heterocycloaklyl; (C₃-C₇)cycloalkyl; O—(C₁-C₈)alkyl; O—(C₁-C₈)alkenyl; O—(C₁-C₈)alkynyl; O-aryl; CN; OH; oxo; halo, C(O)OH; COhalo; O(CO)halo; CF₃; N₃; NO₂; NH₂; NH((C₁-C₈)alkyl); N((C₁-C₈)alkyl)₂; NH(aryl); N(aryl)₂, N((C₁-C₈)alkyl)(aryl); (CO)NH₂; (CO)NH((C₁-C₈)alkyl); (CO)N((C₁-C₈)alkyl)₂; (CO)NH(aryl); (CO)N(aryl)₂; O(CO)NH₂; NHOH; NOH((C₁-C₈)alkyl); NOH(aryl); O(CO)NH((C₁₋₈)alkyl); O(CO)N((C₁-C₈)alkyl)₂; O(CO)NH(aryl); O(CO)N(aryl)₂; CHO; CO(C₁-C₈)alkyl); CO(aryl); C(O)O((C₁-C₈)alkyl); C(O)O(aryl); O(CO)((C₁-C₈)alkyl); O(CO) (aryl); O(CO)O((C₁-C₈)alkyl); O(CO)O(aryl); S—(C₁-C₈)alkyl; S—(C₁-C₈)alkenyl; S—(C₁-C₈)alkynyl; S-aryl; S(O)—(C₁-C₈)alkyl; S(O)—(C₁-C₈)alkenyl; S(O)—(C₁-C₈)alkynyl; and S(O)-aryl; S(O)₂—(C₁-C₈)alkyl; S(O)₂—(C₁-C₈)alkenyl; S(O)₂—(C₁-C₈)alkynyl; and S(O)₂-aryl. One of skill in art can readily choose a suitable substituent based on the stability and pharmacological activity of the compound of the invention.

The term “emulsion” as used herein refers to a suspension of small globules of one liquid (the dispersed phase) in a second liquid (the continuous phase) with which the first will not mix. Accordingly, the term “water-in-oil emulsion” is construed to mean a suspension of small globules of water (the dispersed phase) in an oil solvent (the continuous phase).

The term “peptide” as used herein refers to any compound containing two or more amino acid residues joined by an amide bond formed from the carboxyl group of one amino acid residue and the amino group of the adjacent amino acid residue. The term “peptide” includes oligopeptide, peptide, polypeptide and derivatives thereof, peptide analogs and derivatives thereof, as well as pharmaceutically acceptable salts of these compounds.

The terms “polypeptide” and “protein” are used interchangeably and refer to any polymer of amino acids (dipeptide or greater) linked through peptide bonds or modified peptide bonds, whether produced naturally or synthetically. The term “protein” may refer, in addition, to a complex of two or more polypeptides. Non-limiting examples of proteins include an antibody, an antibody fragment and a peptide aptamer.

The term “oligopeptide” as used herein refers to a peptide containing a relatively small number of amino acid residues; typically around 2 to about 20 amino acids. Examples of oligopeptides include dipeptides, tripeptides, tetrapeptides, and pentapeptides.

The term “synthetic peptide” or “synthetic protein” as used herein refers to man-made molecules that mimic the function and structure of natural peptides or proteins. Synthetic peptides and proteins typically have genetic sequences that are not seen in natural proteins.

The term “stapled peptide” as used herein refers to artificially modified peptide in which the structure is stabilized with one or more artificial molecular bridging (cross links) that connects adjacent turns of α-helices in the peptide.

As used herein, the term “recombinant” refers to a compound or composition produced by human intervention. For example, a “recombinant” nucleic acid or protein molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

The term “biological molecule” refers to any molecule that are created or used by living organisms or cells, or derivatives of such molecules. These molecules may be of natural, synthetic or semisynthetic origin, and includes proteins and nucleic acids.

The term “target molecule” as used herein refers to any molecule with which a biological molecule interacts (e.g. binds), and may for example be a ligand or substrate of the biological molecule. A target molecule may be a peptide, a polypeptide or protein (e.g. a fusion protein), a protein or polypeptide fragment, or functional protein or polypeptide domain.

The term “expression” as used herein refers interchangeably to expression of a gene or gene product, including the encoded protein. A gene is expressed in or by a cell to form an “expression product” such as mRNA or a protein. The expression product itself, e.g. the resulting mRNA or protein, may also be said to be “expressed” by the cell. Expression of a gene may be determined, for example, by measuring the production of messenger RNA (mRNA) transcript levels. Expression of a polypeptide gene product may be determined, for example, by immunoassay using an antibody(ies) that bind with the polypeptide.

The word “substantially” does not exclude “completely” e.g. a composition which is “substantially free” from Y may be completely free from Y. Where necessary, the word “substantially” may be omitted from the definition of the invention.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Unless otherwise indicated, all technical and scientific terms used herein have the same meaning as commonly understood to one of ordinary skill in the art to which the invention belongs.

### Disclosure of Optional Embodiments

Exemplary, non-limiting embodiments of a method for determining resistance of a biological molecule to inhibition by its inhibitor will now be disclosed.

There is provided a method of determining resistance of a biological molecule to inhibition of its interaction with a target molecule by an inhibitor of said biological molecule, the method comprising the steps of:
a) co-compartmentalizing a gene encoding the biological molecule with the target molecule, or a gene encoding the biological molecule with a gene encoding the target molecule into an aqueous droplet disposed within a water-in-oil emulsion, and
b) assaying for a complex comprising the biological molecule and the target molecule upon expression of the gene encoding the biological molecule and the gene encoding the target molecule,
wherein detection of the complex in the presence of the inhibitor indicates that the biological molecule is resistant to inhibition of its interaction with the target molecule by the inhibitor, and
wherein non-detection of the complex in the presence of the inhibitor indicates that the biological molecule is not resistant to inhibition of its interaction with the target molecule by the inhibitor.

There is also provided a method of determining resistance of a biological molecule to inhibition of its interaction with a target molecule by an inhibitor of the biological molecule, the method comprising the steps of:
a) co-compartmentalizing a gene encoding the biological molecule with a gene encoding the target molecule, and the inhibitor into an aqueous droplet disposed within a water-in-oil emulsion, and
b) assaying for a complex comprising the biological molecule and the target molecule upon expression of the gene encoding the biological molecule and the gene encoding the target molecule,
wherein detection of the complex indicates that the biological molecule is resistant to inhibition of its interaction with the target molecule by the inhibitor, and
wherein non-detection of the complex indicates that the biological molecule is not resistant to inhibition of its interaction with the target molecule by the inhibitor.

In one embodiment, the complex comprises the gene encoding the biological molecule of interest. In other words, the complex comprises the expressed biological molecule of interest, the expressed target molecule, and the gene encoding the biological molecule such that a protein-protein-DNA complex is formed. Advantageously, this facilitates selection for and isolation of the gene encoding the biological molecule which exhibits resistance to its inhibitor.

In one embodiment, the gene encoding the biological molecule comprises at least one copy of a p53 response element (RE) fused to the gene encoding the biological molecule. In some embodiments, the gene encoding the biological molecule comprises 2, 3, 4, 5, 10, 15, 20, 25, 30, 40, 50, 60, 70, 80, 90, 100, 2 to 100, 5 to 100, 10 to 100, 20 to 100, 30 to 100, 50 to 100, 75 to 100, 90 to 100, 2 to 90, 2 to 75, 2 to 50, 2 to 30, 2 to 20, 2 to 10, or 2 to 5 copies of a p53 response element (RE) fused to the gene encoding the biological molecule.

In one embodiment, the gene encoding the biological molecule comprises two (2) copies of a p53 response element (RE) fused to the gene encoding the biological molecule.

In one embodiment, the p53 response element is a CONA response element. Other exemplary response elements that may be used are known to those skilled in the art, and include, but are not limited to, p21 and puma response elements.

In this way, p53 can act as a linker protein to link the gene encoding the biological molecule (or a variant thereof) with the biological molecule (or a variant thereof) that is expressed in protein form from the gene, thus forming a protein-protein-DNA complex in the presence of an inhibitor of the biological molecule. In other words, the p53 can act as a linker between the genotype (gene encoding the biological molecule or a variant thereof) and the phenotype of the biological molecule (or variant thereof) in formation of the complex in presence of the inhibitor.

The disclosed method may be applied to other protein-protein interactions (apart from interaction between p53 and HDM2) by generating a fusion protein comprising a protein of interest and p53. This can be added to emulsion compartments in purified form, or expressed inside emulsion compartments from a gene construct encoding the fusion protein. A gene construct comprising two or more tandem copies of a p53 response element and the biological molecule are also introduced into the emulsion compartments.

In some embodiments, the emulsion comprises a plurality of the aqueous droplets. Preferably, each aqueous droplet comprises a single variant of the gene encoding the biological molecule of interest.

The biological molecule may be selected from the group consisting of an amino acid, a peptide, a protein and combinations thereof. The peptide may be a polypeptide, an oligopeptide, a stapled peptide, or a synthetic peptide. The protein may be a mini-protein, a recombinant protein or a protein complex.

The polypeptides of the invention may be “free-standing”, i.e. not part of or fused to other amino acids or polypeptides or they may be comprised within a larger polypeptide of which they form a part or region. Hence, fusion proteins incorporating the polypeptides described herein are contemplated in the present invention. For example, it is often advantageous to include one or more additional amino acid sequences which may contain secretory or leader sequences, pro-sequences, or sequences which aid in for instance detection, expression, separation or purification of the protein or to endow the protein with additional properties as desired such as higher protein stability. Examples of potential fusion partners include epitope tags (short peptide sequences for which a specific antibody is available), polyethylene glycol, beta-galactosidase, luciferase, a polyhistidine tag, glutathione S transferase (GST), a secretion signal peptide and a label, which may be, for instance, bioactive, radioactive, enzymatic or fluorescent, or an antibody.

A fusion protein may also be engineered to contain a cleavage site located between the sequence of a polypeptide of the invention and the sequence of a heterologous protein/polypeptide sequence so that the polypeptide may be cleaved and purified away from the heterologous protein/polypeptide sequence. By a “heterologous protein” and “heterologous polypeptide sequence”, it is meant a protein or an amino acid sequence which, in nature, is not found in association with a polypeptide of the invention.

In one embodiment, the biological molecule is a protein. The protein may be an enzyme. In one embodiment, the protein is HDM2 ubiquitin ligase (“HDM2”). In another embodiment, the protein is a homologue of HDM2, for example HDMX or HDM4. In another embodiment, the protein may be selected from the group consisting of tumor suppressor p53-binding proteins, such as tumor suppressor p53-binding protein 1 (TP53BP1) and tumor suppressor p53-binding protein 2 (TP53BP2). Other proteins that may be used as the biological protein to form a protein-protein or protein-protein-DNA complex in the methods disclosed herein can be found on publicly available databases, such as the UniProt database (http://www.uniprot.org/).

The target molecule may also be selected from the group consisting of an amino acid, a peptide, a protein and combinations thereof. The peptide may be a polypeptide, an oligopeptide, a stapled peptide and a synthetic peptide. The protein may be a mini-protein, a recombinant protein and a protein complex.

In one embodiment, the target molecule is a protein. In one embodiment, the protein is a p53 tumor suppressor protein (“p53”).

Hence, in one embodiment, the biological molecule of interest whose resistance to its inhibitor is to be determined is HDM2, and the target molecule to which HDM2 binds is its substrate, p53.

In another embodiment, the target molecule may comprise a fusion protein. The fusion protein may comprise a p53 fused to a protein (e.g. protein Y) with which the biological molecule interacts. Hence, in such an embodiment, the protein-protein-DNA complex that is formed comprises the biological molecule, the fusion protein (e.g. comprising p53 and protein Y), and the gene encoding the biological molecule. As discussed above, the fusion protein (e.g. comprising p53 and protein Y) may be included in the compartments within the emulsion in purified form, or expressed inside the compartments from a gene construct encoding the fusion protein. A gene construct comprising two or more tandem copies of a p53 response element and the biological molecule may also be included in the compartments within the emulsion.

The inhibitor may be a small organic molecule, a peptide or a protein. The peptide may be selected from the group consisting of a polypeptide, an oligopeptide, a stapled peptide and a synthetic peptide. The protein may be selected from the group consisting of a mini-protein, a recombinant protein and a protein complex. An inhibitor may also be a ribozyme or an antibody. Analogues of the inhibitor are also included herein.

A “small molecule” is an organic (having at least one carbon atom) or inorganic (having no carbon atoms) compound that has a molecular weight that is sufficiently low (typically <900 Daltons) to allow the small molecule to rapidly diffuse across cell membranes so that they can reach intracellular sites of action. Preferably, the inhibitor is a small organic molecule. Exemplary small organic molecules include, but are not limited to, pharmaceuticals (i.e. drugs, such as anti-cancer drugs), sugars, fatty acids, steroids, saccharides, purines, pyrimidines, derivatives, structural analogs, or combinations thereof.

An inhibitor of the HDM2 ubiquitin ligase includes any molecule capable of decreasing the activity of the ligase, for example by interfering with interaction of the ligase with another molecule, such as its substrate, e.g. p53. One such inhibitor is the small organic molecule Nutlin or analogues thereof. Analogues of Nutlin may be “Nutlin-like” molecules that have the same mode of action as Nutlin. Examples of Nutlin include but are not limited to Nutlin 1, Nutlin 2, and Nutlin 3. Preferably, the Nutlin 3 is Nutlin 3A. Examples of Nutlin-like molecules include but are not limited to RG7112, MI-219, AM-8553 and BZD-17. Other small organic molecules that may inhibit HDM2 ubiquitin ligase include but are not limited to MI-5, MI-17, MI-63, MI-219, MI-888, N,N-dibenzylcinnamoyl amide, N,N-dibenzylbenzamide, 1,4-benzodiazepine-2,5-dione (EZD), WK298 and WK23.

Hence, in one embodiment, the disclosed method may be used for determining the resistance of HDM2 (or a variant thereof) to inhibition of its interaction with its target molecule, p53, by the inhibitor Nutlin.

In another embodiment, the disclosed method may be used for determining the resistance of steroid receptors (e.g. estrogen receptors) to inhibition of its interaction with its target proteins (e.g. p300, CEP, SP1 etc.) by an inhibitor. The inhibitor may be a small molecule inhibitor, such as tamoxifen.

The inhibitor may be present at a concentration that is capable of inhibiting the interaction of a wild-type form of the biological molecule with the target molecule. The concentration of the inhibitor may be, for example, at least about 1 μM, at least about 2 μM, at least about 3 μM, at least about 4 μM, at least about 5 μM, at least about 6 μM, at least about 7 μM, at least about 8 μM, at least about 9 μM, at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 110 μM, at least about 120 μM, at least about 130 μM, at least about 140 μM, or at least about 150 μM. The exact concentration will vary from one biological molecule-target molecule pair to another. For any given case, an appropriate concentration may be determined by one of ordinary skill in the art using routine experimentation.

The co-compartmentalization of a gene encoding the biological molecule of interest with a gene encoding its target molecule and its inhibitor in step a) of the disclosed method may be carried out using methods known in the art for forming emulsions. For example, in order to emulsify the aqueous (dispersed) phase into the oil (continuous) phase to give a water-in-oil emulsion, the aqueous phase and the oil phase are mixed in the presence of an emulsifying agent of the water-in-oil type. Any conventional water-in-oil emulsifying agent can be used, such as mineral oil, hexadecyl sodium phthalate, sorbitan monooleate (e.g. Span 80), sorbitan monostearate, polysorbitan (e.g. Tween 80), cetyl or stearyl sodium phthalate, metal soaps, and the like.

In one embodiment, step a) of the disclosed method comprises co-compartmentalizing the gene encoding the biological molecule and the target molecule into an aqueous droplet disposed within a water-in-oil emulsion. In other words, the target molecule is in the form of an expressed product, i.e. a protein, and may be in purified form. In such an embodiment, it may not be necessary to include the gene encoding the target molecule in the aqueous droplet.

Alternatively, in one embodiment, step a) of the disclosed method comprises co-compartmentalizing the gene encoding the biological molecule and the gene encoding the target molecule into an aqueous droplet disposed within a water-in-oil emulsion. In this embodiment, any complex between the biological molecule and the target molecule may be formed upon expression of the gene encoding the biological molecule and the gene encoding the target molecule.

In one embodiment, step a) comprises co-compartmentalizing an inhibitor with the gene encoding the biological molecule, and the target molecule or the gene encoding the target molecule into an aqueous droplet disposed within a water-in-oil emulsion.

In one embodiment, step b) comprises assaying for the complex comprising the biological molecule and the target molecule upon expression of the gene encoding the biological molecule and/or the gene encoding the target molecule after rupturing the aqueous droplet and contacting the contents thereof with the inhibitor.

In one embodiment, step b) comprises assaying for the complex by “off-rate” selection. Methods for performing off-rate selection have been described, for example in Ylera F et al. (2013). Anal Biochem. 2013 Oct. 15; 441(2):208-13.

Step b) of the disclosed method may also comprise rupturing the aqueous droplets and contacting the contents thereof with a detectable label capable of binding to a complex comprising the expressed biological molecule of interest, its expressed target molecule, and the gene encoding the biological molecule, to assay for any such complex that may have been formed. The “detectable label” may be a reporter molecule or enzyme that is capable of generating a measurable signal and is covalently or non-covalently joined to a polynucleotide or polypeptide. Exemplary detectable labels include, but are not limited to, magnetic bead labels, antibodies, radioisotope labels, luminescent labels, fluorescent labels, enzyme labels, colloidal metal labels, colored glass bead labels, colored latex bead labels, carbon black labels, or combinations thereof. The label may be a “direct” label that is coupled (i.e. physically linked) to a component of the complex to be detected, or an “indirect” label of the complex by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In vitro detection of the polypeptides or variants or fragments thereof of the present invention may be achieved using a variety of techniques including ELISA (enzyme linked immunosorbent assay), Western blotting; immunoprecipitation and immunofluorescence. Such techniques are commonly used by those of skill in the art.

In some embodiments, the disclosed method further comprises step c) of amplifying the gene encoding the biological molecule in the complex that has been detected and detecting the amplified gene. The term “amplification” as used herein refers to the production of additional copies of a nucleic acid. Amplification may be carried out using polymerase chain reaction (PCR) technologies or other nucleic acid amplification technologies well known in the art.

In further embodiments, the disclosed method comprises identifying a mutation/(s) in the gene encoding the biological molecule in the complex that has been detected. Methods for identification of a mutation/(s) present in a gene are well known in the art, and include for example, sequence analysis.

In yet further embodiments, the disclosed method comprises analyzing in silico the interaction between the biological molecule and/or the target molecule and/or the inhibitor to determine the mechanism of resistance of the biological molecule to inhibition of its interaction with the target molecule by the inhibitor.

Steps a), b) and c) of the disclosed method may be repeated at least once, at least twice, at least three times, at least four times, at least five times, at least six times, at least seven times, at least eight times, at least nine times, at least 10 times, more than 10 times, more than 15 times, more than 20 times, more than 25 times, more than 30 times, more than 35 times, more than 40 times, more than 45 times, more than 50 times, more than 55 times, more than 60 times, more than 65 times, more than 70 times, more than 75 times, more than 80 times, more than 80 times, more than 90 times, more than 90 times, or more than 100 times.

In one embodiment, the disclosed method is conducted in vitro. Advantageously, conducting the disclosed method in vitro enables the application of stringent selection pressures, thus enabling identification of mutant/variant forms of the biological molecule with exceptionally strong phenotypes.

There is also provided a kit for use in a method as described above, wherein the kit comprises means to co-compartmentalize the gene encoding the biological molecule with the target molecule, or the gene encoding the biological molecule with a gene encoding the target molecule, and optionally the inhibitor, into aqueous droplets disposed within a water-in-oil emulsion, and means to detect the complex comprising the biological molecule and the target molecule upon expression of the gene encoding the biological molecule and/or the gene encoding said target molecule. The means for co-compartmentalizing the gene encoding the biological molecule with the gene encoding the target molecule, and optionally the inhibitor, into aqueous droplets, may include buffers, emulsifying agents etc. to form water-in-oil emulsions. The means for detecting any complex that may have been formed between the biological molecule and the target molecule upon expression of the gene encoding the biological molecule and/or the gene encoding said target molecule include, for example, a detectable label as described above.

In some embodiments, the kit comprises means to detect the gene encoding the biological molecule also present in the complex.

The reagents that are suitable for detecting the complex may include reagents that may incorporate a detectable label, such as a fluorophore, radioactive moiety, enzyme, biotin/avidin label, chromophore, chemiluminescent label, or the like, or the kits may include reagents for labeling the nucleic acids for detecting the presence or absence of the gene encoding the biological molecule as described herein. The kit may further comprise reagents including, but are not limited to reagents for isolating peptides/proteins from samples, reagents for positive or negative controls and reagents for assays as described herein. For example, the kits may include reagents used in the Experimental section below.

The kit may further comprise instructions that may be provided in paper form or in computer-readable form, such as a disc, CD, DVD or the like. The kits may optionally include quality control reagents, such as sensitivity panels, calibrators, and positive controls.

The kits can optionally include other reagents required to conduct a diagnostic or prognostic assay or facilitate quality control evaluations, such as buffers, salts, enzymes, enzyme co-factors, substrates, detection reagents, and the like. Other components, such as buffers and solutions for the isolation and/or treatment of a test sample (e.g. pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. The kit further can include containers for holding or storing a sample (e.g. a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

Also provided herein are mutated ubiquitin ligase polypeptides comprising at least one mutation. The mutation may be selected from the group consisting of E69A, D225G, V241A, V280A, K344R, E390G, V426A, Q442R, M459T, Q24R, M62V, E124G, C461Y, T16A, P20L, L254F, N309T, G443D, H457R, L82P, and combinations thereof. In one embodiment, the mutated ubiquitin ligase polypeptide comprises the mutations E69A, D225G, V241A, V280A, K344R, E390G, V426A, Q442R and M459T. In another embodiment, the mutated HDM2 ubiquitin ligase polypeptide comprises the mutations Q24R, M62V, E124G and C461Y. In a further embodiment, the mutated HDM2 ubiquitin ligase polypeptide comprises the mutations T16A, P20L, L254F, V280A, N309T, G443D and H457R. In yet other embodiments, the mutated HDM2 ubiquitin ligase polypeptide comprises the mutation L82P.

There is further provided a DNA molecule encoding the mutated HDM2 ubiquitin ligase polypeptide as described above.

The disclosed method may be used to assess the receptiveness of a cancer patient to treatment with an anti-cancer drug. Thus, there is provided a prognostic method for determining the receptiveness of a cancer patient to treatment with an anti-cancer drug capable of inhibiting the interaction of HDM2 ubiquitin ligase with p53 tumor suppressor protein, the method comprising the step of:
comparing a gene encoding the HDM2 ubiquitin ligase derived from a sample of the patient against a plurality of HDM2 ubiquitin ligase genes that have been determined to be resistant to the anti-cancer drug by the method as described above; wherein identification of a match between the gene of the patient to at least one gene in the plurality of HDM2 ubiquitin ligase genes that have been determined to be resistant to the anti-cancer drug indicates that the cancer patient may not be receptive to treatment with the anti-cancer drug.

In one embodiment, the anti-cancer drug is a Nutlin or analogues thereof (e.g. Nutlin-like molecules). The Nutlin may be selected from the group consisting of Nutlin 1, Nutlin 2 and Nutlin 3. In one embodiment, the Nutlin 3 is Nutlin 3A. Nutlin-like molecules may be selected from the group consisting of RG7112, MI-219, AM-8553 and BZD-17. In another embodiment, the Nutlin analogue is RG7112.

In one embodiment, the anti-cancer drug is a stapled peptide. In one embodiment, the stapled peptide targets the same interaction and/or the same site on the HDM2 as Nutlin.

The sample used in the disclosed prognostic method may be a biological sample such as tissues, cells, whole blood, blood fluids (e.g. serum and plasma), lymph and cystic fluids, sputum, stool, tears, mucus, hair, skin, ascitic fluid, cystic fluid, urine, nipple exudates, nipple aspirates, sections of tissues such as biopsy and autopsy samples, frozen sections taken for histologic purposes, archival samples, and explants, primary and transformed cell cultures derived from patient tissues. The sample may be untreated, treated, diluted or concentrated from a patient, and may comprise an extract from a cell, chromosome, organelle, or membrane isolated from a cell; a cell; genomic DNA, RNA, or cDNA, in solution or bound to a substrate; etc.

The cancer of the patient who may be subjected to the disclosed prognostic method includes but is not limited to retinoblastoma, blood malignancies (e.g. leukaemia), biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment, the cancer is leukaemia or melanoma.

The disclosed method may also be used to select for a variant form of a biological molecule of interest that is resistant to inhibition of its interaction with a target molecule by an inhibitor of the biological molecule. Thus, there is provided a method for selecting a variant form of a biological molecule that is resistant to inhibition of its interaction with a target molecule by an inhibitor of the biological molecule, comprising the steps of providing a plurality of randomly mutated genes encoding the biological molecule, and determining resistance of the biological molecule to inhibition of its interaction with the target molecule by the inhibitor using the method as described above. The plurality of randomly mutated genes encoding the biological molecule may be generated using methods known in the art, for example by passing cloned genes through mutator strains, by “error-prone” PCR mutagenesis, by rolling circle error-prone PCR, or by saturation mutagenesis.

The plurality of randomly mutated genes encoding the biological molecule may comprise at least 10⁴ randomly mutated genes encoding the biological molecule, at least 10⁵ randomly mutated genes encoding the biological molecule, at least 10⁶ randomly mutated genes encoding the biological molecule, at least 10⁷ randomly mutated genes encoding the biological molecule, at least 10⁸ randomly mutated genes encoding the biological molecule, at least 10⁹ randomly mutated genes encoding the biological molecule, at least 10¹⁰ randomly mutated genes encoding the biological molecule, at least 10¹¹ randomly mutated genes encoding the biological molecule, at least 10¹² randomly mutated genes encoding the biological molecule, at least 10¹³ randomly mutated genes encoding the biological molecule, at least 10¹⁴ randomly mutated genes encoding the biological molecule, or at least 10¹⁵ randomly mutated genes encoding the biological molecule. Preferably, the plurality of randomly mutated genes encoding the biological molecule comprises at least 10⁷ randomly mutated genes encoding the biological molecule, at least 10⁸ randomly mutated genes encoding the biological molecule, at least 10⁹ randomly mutated genes encoding the biological molecule, or at least 10¹⁰ randomly mutated genes encoding the biological molecule.

There is also provided a kit for use in the method of selecting a variant form as described above, comprising means for generating the plurality of randomly mutated genes encoding the biological molecule. The kit may further comprise means for co-compartmentalizing a randomly mutated gene encoding the biological molecule, a gene encoding a target molecule of the biological molecule, and an inhibitor of the interaction of the biological molecule with the target molecule into an aqueous droplet disposed within a water-in-oil emulsion as described above, as well as means for detecting a complex comprising the biological molecule and the target molecule upon expression of the randomly mutated gene encoding the biological molecule and the gene encoding the target molecule as described above.

There is further provided a method of restoring the inhibitory activity of a drug on the interaction of a biological molecule with a target molecule, the method comprising the steps of:
i) identifying a variant of the biological molecule that is resistant to inhibition of its interaction with the target molecule by the drug, using the method as described above; and
ii) modifying the drug to restore its inhibitory activity on the interaction of the biological molecule with the target molecule.

Step i) may comprise determining the mechanism of resistance of the biological molecule to inhibition of its interaction with its target molecule by the drug. The method may also comprise analyzing the structure of the biological molecule determined to be resistant to inhibition of its interaction with the target molecule by the inhibitor. The structural analysis may be carried out using methods well known in the art, such as nuclear magnetic resonance (NMR) and X-ray crystallography. Determination of the mechanism of resistance and analysis of the structure of the biological molecule having the resistant phenotype provides information to facilitate iterative refinement of the drug so as to improve their functionality and/or restore their inhibitory effect on the biological molecule. The information may also be useful for assessing alternative therapeutic candidates for overcoming or circumventing the drug resistance, and for predicting future resistant phenotypes that may arise in a clinical setting.

The modification of the drug in step b) may comprise modifying the drug to overcome the mechanism of resistance such that the inhibitory activity of the drug on the interaction of the biological molecule with its target molecule can be restored.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 (A) shows a schematic depicting a complex formed between HA-tagged HDM2, p53 and DNA captured on beads coated with anti-HA antibody. Arrows represent PCR primers for quantifying the captured DNA or amplifying HDM2 genes during selection. (B) shows a bar graph depicting the results of a real-time PCR assay measuring the complex formation (shown in FIG. 1A) in the presence of Nutlin (at concentrations of 0, 10, and 100 μM). The values on the y-axis indicate the fold increase over HDM2 gene control without any p53 response element (2CONA) appended. The values represent mean+/−SD (n=2), *p<0.05. (C) shows a Western blot of p53 captured by immobilised HDM2 and the effect of Nutlin (at a concentration of 10 μM).

FIG. 2 shows a schematic depicting the selection of Nutlin-resistant HDM2 by in vitro compartmentalization (IVC). 1. HDM2 expression constructs appended with 2CONA p53 response element (“RE”) and HA-tag coding sequence (“HA”) and p53 expression construct (“p53”) are segregated into aqueous emulsion compartments along with Nutlin (“N” orbs). Protein expression occurs within the compartments. Nutlin inhibition of HDM2 results in no HDM2-p53-DNA complex formation (left bubble), whereas resistant HDM2 can form the complex (right bubble). 2. The emulsion is broken and complexes captured with anti-HA antibody. 3. DNA encoding resistant HDM2 variants is amplified by PCR. 4. Selectants are further evaluated by secondary pulldown assay or subjected to further rounds of selection.

FIG. 3 (A) depicts the in vitro pulldown assay showing reduced inhibition by Nutlin (10 μM) to binding of p53 for indicated parental HDM2 variants. (B) and (C) depict the analysis of individual mutations derived from parental clones 5.9 and 5.14, respectively. The analysis showed some of these mutants to confer Nutlin-resistance. * Indicates no Nutlin treatment.

FIG. 4 depicts the results of inhibition of selected variants in p53-null H1299 cells. (A) shows the results on H1299 cells co-transfected with either p53 alone or p53 and the indicated HDM2 variants. p53 function was measured by reporter gene activity in presence of Nutlin (at concentrations of 0, 2, or 5 μM). The values represent mean+/−SD. (B) As in A, with Nutlin-induced increases plotted relative to the base line value of inhibition in the absence of drug treatment (set to 1). The values represent mean+/−SD. (C) Western blot showing expression levels of HDM2 variants and p53 in H1299 cells.

FIG. 5 shows the results of inhibition of selected HDM2 variants in p53/HDM2-null DKO cells. (A) Wild-type and indicated N-terminal domain mutants were co-transfected with p53 and p53 reporter gene activity was measured in the presence of Nutlin (at concentrations of 0, 2, 5, or 10 μM). Representative data from one experiment are shown. (B) Wild-type and indicated acidic (V280A), zinc finger (C308Y, N309T, C322R) and RING (G443D) domain mutants were co-transfected with p53 and p53 reporter gene activity was measured in the presence of Nutlin (at concentrations of 0, 2, 5, or 10 μM). Representative data from one experiment are shown. (C) Representative Western Blots showing expression levels of HDM2 variants and p53 DKO cells.

FIG. 6 depicts a bar graph showing the results of Fluorescent 2-Hybrid (F2H) assay of p53 with wild-type (WT) HDM22, mutant Q24R and M62A. The F2H assay investigates the interaction of p53 (bait) with wild-type (WT) HDM2, mutant Q24R and M62A (preys). The F2H assay measures the interaction between two proteins as ratio of cells showing co-localization of bait and prey at the nuclear F2H interaction platform, to cells not showing this co-localization. Titration of Nutlin on to BHK cells co-transfected with GFP-p53 and RFP-HDM2 (wt) immediately resulted in declined percentage of co-localization. In contrast, p53 interaction with HDM2 mutants Q24R and M62A upon Nutlin treatment was clearly less reduced, indicating Nutlin resistance. Graph bars show means of normalized interaction values (in %) ±s.e.m. from three to five independent experiments. n.s. no significance, *p<0.05, **p<0.01.

FIG. 7 shows images of the Q24R mutation in the HDM2 lid region. The Q24R mutation in the HDM2 lid region was predicted to enhance affinity for p53 but not Nutlin. (A) Molecular simulations indicate mutation of Q24 to arginine (right structure) leads to repulsion of proximal K51 and stabilization of E28 in p53 (circled, right structure) through charge-charge interaction. This additional stabilization is not seen in wild-type HDM2 bound to p53 (circled, left structure). (B) Q24 is seen to make no significant contact with Nutlin (left structure) and mutation to arginine (right structure) did not result in any additional differences.

FIG. 8 depicts images of the packing interactions between the HDM2 p53-binding domain and Nutlin. The M62A mutant in the HDM2 p53-binding domain selectively resulted in loss of Nutlin binding. (A) Simulations indicate loss of significant packing interactions with Nutlin (left structure, circled) when M62 is mutated to alanine (right structure, circled). (B) Packing interactions with p53 (left structure, circled) are seen to be minimally disrupted by M62A mutation (right structure, circled).

FIG. 9 shows that the mutation V280A in acidic domain resulted in reduced interaction with p53 DNA binding domain. (A) shows models of the native (left) and V280A mutant (right) peptides docked to the p53 DNA binding domain. Residues set as “active” in the Haddock server run (details in Materials and Methods) are shown as sticks (red and yellow for p53 and blue for peptide). Residues of p53 known to make direct contact with DNA are colored in yellow. V280 and A280 are shown in green. In the native case, V280 appears to make more contacts with p53 than A280 in the mutated peptide. (B) In vitro pulldown assay shows reduced interaction between HDM2 V280A and full-length (comprising both N-terminal and DNA binding domain HDM2 interaction sites) and N-terminally truncated (Δ133) p53 (DNA binding domain HDM2 interaction site only). The expression level of the V280A mutant is seen to be increased compared to wild-type HDM2.

FIG. 10 depicts a table indicating the mutations present in HDM2 selectants displaying in vitro Nutlin resistance. The upper schematic shows the domain architecture of the HDM2 gene.

FIG. 11 shows the results of the pulldown assay investigating the resistance of the HDM2 variants. In the presence of Nutlin (10 μM), no significant increase in p53 binding was observed for mutant HDM2 compared to wild-type.

FIG. 12 shows a graph depicting the results of inhibition of HDM2 variants in p53/HDM2-null DKO cells. DKO cells were co-transfected with either p53 alone or p53 and the indicated HDM2 variants. p53 function was measured by reporter gene activity in the presence of the indicated amounts of Nutlin. Activity was expressed as percentage of reporter gene transactivation seen with wild-type HDM2 (set to 100, indicated by dotted line). The values represent mean±SD from two to three independent experiments, *p<0.05, **p<0.05, ***p<0.005. (A) Wild-type and indicated N-terminal domain mutants were co-transfected with p53 and p53 reporter gene activity was measured in the presence of Nutlin (at concentrations of 0, 2, 5, or 10 μM). (B) shows a graph depicting the result of inhibition of HDM2 variant L82P in p53/HDM2-null DKO cells.

FIG. 13 depicts time lapse images which indicate persistence of mutant HDM2-p53 complex in presence of Nutlin using F2H assay. Green dot shows p53 bound to DNA. Co-localised red dot shows HDM2 in complex with p53. Arrows indicate last time point where HDM2 (wild-type or indicated mutant) was present in complex. Time is indicated in minutes.

FIG. 14 depicts the distribution of energies of interactions (enthalpies) of p53 (top) and Nutlin (bottom) with wild-type and the mutants Q24R and M62A. The enthalpies were computed using standard protocols as outlined in [16].

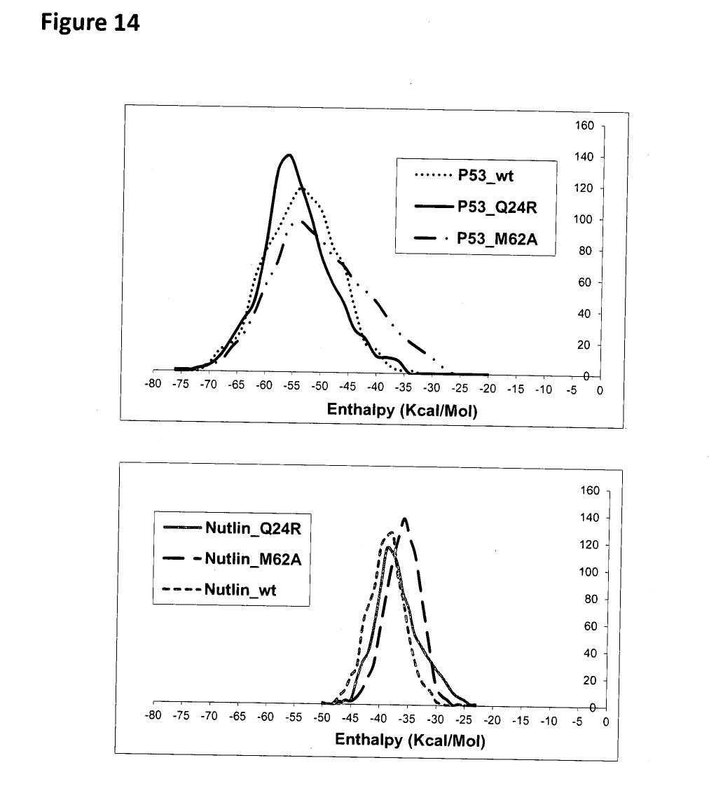

FIG. 15 shows that Nutlin-resistant HDM2 variants are inhibited by stapled peptides. (A) In vitro pull-down assay shows that Nutlin (10 μM) inhibits p53-HMD2 interaction but was less effective for the Q24R and M62A HDM2 variants (top row). Stapled peptides PM2 and MO11 (10 μM) showed inhibition of p53 binding to both WT and mutant HDM2 (second row). 10% of respective HDM2 inputs loaded. PM2CON was used as negative control stapled peptide. (B) Sequences of stapled peptides PM2 and MO11. The residues at positions 3, 7, 9 of PM2 were mutated to alanine in PM2CON. “X” denotes staple tethering sites. A chlorine atom is added to the C6 position of W7 in MO11.

FIG. 16 shows that stapled peptides inhibit wild-type and mutant HDM2 function in p53/MDM2-null DKO cells. (A) Wild-type and indicated HDM2 mutants were co-transfected with p53 and p53 reporter gene activity (expressed as percentage of activity observed with p53-only transfection) was measured in the presence of Nutlin (at concentrations of 0, 5, or 10 μM) and the stapled peptides PM2CON, PM2 and MO11 (at concentrations of 10 or 20 μM). Data represents mean±SD (n=2). (B) Western blots showing expression levels of HDM2 variants and p53 co-transfected into DKO cells.

FIG. 17 shows that stapled peptides inhibit wild-type and mutant HDM2 function in HCT116 p53^{+/+} cells as measured by transcriptional response of p53-regulated genes. The concentration dependent effect of Nutlin (0-10 μM) or DMSO vehicle and stapled peptide PM2 or inactive control peptide (CP) on expression of p21, 14-3-3σ and gadd45α genes determined at 24 h post-transfection of HDM2 (WT) and indicated HDM2 mutants. The data show the fold change in gene expression by RT-qPCR (Ct method) compared to vehicle or control peptide treated cells transfected with pcDNA empty vector. * p<0.05, ** p<0.01, *** p<0.001 (WT versus respective mutant). Data represents mean±SEM (n=2).

FIG. 18 shows the differential binding of stapled peptide versus Nutlin to HDM2-M62A. Competition titrations of MO11 and Nutlin were carried out against FAM-labelled p53-binding peptide 12.1 for binding to wild-type (top) and M62A (bottom) HDM2 mutant. The values represent mean+/−SD (n=2).

FIG. 19 shows that the direct interaction between p53 and HDM2 N-terminal domain was inhibited by stapled peptides. F2H assay was carried out to investigate the interaction of p53 (bait) with wild-type HDM2 (WT), mutant Q24R and M62A (preys). The F2H assay measures the interaction between two proteins as ratio of cells showing co-localization of bait and prey at the nuclear F2H interaction platform, to cells not showing this co-localization. (A) Titration of Nutlin resulted in increased dissociation of WT-p53 complex compared to Q24R/M62Ap53 complexes. (B) and (C) In contrast, the stapled peptides displayed equivalent potency on WT and mutant HDM2. The data points represent normalized interaction values expressed as percentage interactions observed in the absence of treatment±SD (n=4). * p<0.05, ** p<0.01, *** p<0.005 (WT vs M62A, blue asterisks. WT vs Q24R, red asterisks).

FIG. 20 shows that the M62A mutation in HDM2 does not perturb binding of the stapled peptide PM2. Left: Simulations (see Materials and Methods) indicate packing interactions between M62 (yellow) and hydrophobic staple (cyan) of PM2. Right: Mutation to alanine resulted in loss of these interactions, but numerous other interactions persist between PM2 and hydrophobic cleft of HDM2. The peptidic component of PM2 is depicted in magenta.

FIG. 21 depicts molecular simulations showing the negative impact of the P20L and Q24R mutations on the docking of Nutlin to the HDM2 N-terminal domain. (A) Space-filling (left) and ribbon (right) depictions of Nutlin binding the main (arrowed) and secondary (circled) binding sites of HDM2. The p53-peptide (cyan) binding to the main site is overlaid. (B) Space-filling model of the HDM2 N-terminal domain showing migration of the lid region when P20 (left) was mutated to leucine (right, L20 sphere). The arrow depicts the main Nutlin binding site. (C) Mutation of Q24 (left) to arginine (right) resulted in extensive hydrogen bond network with E23 and Y100 and likely occlusion of the secondary Nutlin binding site.

FIG. 22 shows a table depicting the apparent K_{d} values of indicated ligands for HDM2 mutants determined by competitive fluorescence anisotropy titrations. The values represent mean±SD (n=2-4).

FIG. 23 shows a table on the energetic contribution to the differences in the binding free energies (ΔG_{Binding}) between wild type HDM2 and alanine mutant variants for binding to the indicated ligands. Residues contributing >2 kcal/mol are italicized and underlined, and are further depicted in Venn diagram below the table.

FIG. 24 shows the expression levels of HA-tagged wild-type (WT) and indicated HDM2 mutants transfected into HCT116 cells and treated with either Nutlin or stapled peptides PM2CON and PM2 as indicated.

FIG. 25 shows the energetic contribution (kcal/mol) of indicated HDM2 residues to binding of p53 peptide, Nutlin, and stapled peptide PM2 as determined by computational alanine scanning (see Materials and Methods).

### EXAMPLES

Non-limiting examples of the invention, including the best mode, and a comparative example will be further described in greater detail by reference to specific Examples, which should not be construed as in any way limiting the scope of the invention.

### Example 1

### Molecular Mechanisms of HDM2 Resistance

The p53 tumor suppressor functions as a master regulator of cell fate [1,2] and is commonly mutated in cancer [3,4,5,6]. Its pro-apoptotic activity is negatively regulated by HDM2, the ubiquitin-ligase that binds to p53 and targets it for proteosomal degradation [7,8,9,10]. Approximately 50% of cancers harbor wild-type p53, and elevation of p53 levels in these cancers by targeted disruption of the HDM2-p53 complex represents an attractive therapeutic modality [89,38]. Numerous agents including peptides, stapled peptides mini-proteins, and small molecules have been described which bind to the p53-binding pocket in the N-terminal domain of HDM2 [38,90,91,92]. Occlusion of the p53 binding pocket results in rapid elevation of p53 levels, with the attendant downstream expression of proteins eliciting cell-cycle arrest and/or cell death.

Pharmacological modulation of p53 activity is an attractive therapeutic strategy in cancers with wild-type p53. The small molecule Nutlin-3A (hereinafter referred to as “Nutlin”), presently in clinical trials, competitively binds to HDM2, a key negative regulator of p53 and blocks its activity. Nutlin binds to the p53-binding pocket in the N-terminal domain of HDM2 by mimicking core interactions of residues in the p53 transactivation domain that interact with the pocket [93]. It is presently in advanced preclinical development and clinical trials for the treatment of retinoblastoma and blood malignancies with wild-type p53 status [94,95].

Mutant HDM2 has been previously identified in some tumour samples [11,22]. Furthermore, HDM2 gene amplification and over-production in cancer [13,14], and correlation with poor response to therapy [15], suggest that HDM2 mutation could render cells recalcitrant to Nutlin therapy. To investigate this possibility in a targeted manner, it would therefore be desirable, to interrogate large numbers of mutated HDM2 variants for a Nutlin-resistance phenotype, wherein the interaction with p53 is not attenuated by the drug [16].

Using in vitro selection, the emergence of resistance was simulated by evolving HDM2 variants capable of binding p53 in the presence of Nutlin concentrations that inhibit the wild-type HDM2-p53 interaction. The in vitro phenotypes were also seen in ex vivo assays, where Nutlin-dependent activation of p53 transactivation function was significantly reduced in cells co-expressing the selected HDM2 variants. Mutations conferring drug resistance were not confined to the N-terminal p53/Nutlin-binding domain, and were additionally seen in the central acidic, zinc finger and RING domains. The spectrum of HDM2 variants identified sheds further insight into the interaction of HDM2 with both Nutlin and p53, and highlights pathways to resistance which can manifest in the clinic.

### Materials and Methods

Unless otherwise specified, all oligonucleotides used in this work were from 1st Base (Singapore), restriction enzymes from NEB and chemical reagents from Sigma. Nutlin-3A was from Calbiochem.

Primers

| | |
|---|---|
| petF3conA-Rlink: | 5′-GTGACTCAGCGGACATGCCCGGACATGCCCCAGGTGCGGTTGCTGGCGCCTAT-3′ |
| | (SEQ ID NO: 1) |
| petF4conA-Flink: | 5′-GCTGAGTCACGGGCATGTCCGGGCATGTCCGATGCGTCCGGCGTAGAGGATCG-3′ |
| | (SEQ ID NO: 2) |
| petF2: | 5′-CATCGGTGATGTOGGCGAT-3′ |
| | (SEQ ID NO: 3) |
| petR: | 5′-CGGATATAGTTCCTCCTTTCAGCA-3′ |
| | (SEQ ID NO: 4) |
| Hdm2-Nde1: | 5′-CACAACATATGTGCAATACCAACATGTCTGTACC-3′ |
| | (SEQ ID NO: 5) |
| Hdm2-HA-BamH1: | 5′-GCTCTGGATCCTTAAGCGTAATCTGGAACATCGTATGGGTAGGGGAAATAAGTTA-3′ |
| | (SEQ ID NO: 6) |
| INF-Hdm2-cmvF: | 5′-CGAACCTAAAAACAAATGTGCAATACCAACATGTCTGTAC-3′ |
| | (SEQ ID NO: 7) |
| INF-HA-cmvRcor: | 5′-TTATAGACAGGTCAACTAAGCGTAATCTGGAAC-3′ |
| | (SEQ ID NO: 8) |
| mdm2-T16A-QC1: | 5′-GATGGTGCTGTAACCGCCTCACAGATTCCAG-3′ |
| | (SEQ ID NO: 9) |
| mdm2-T16A-QC2: | 5′-CTGGAATCTGTGAGGCGGTTACAGCACCATC-3′ |
| | (SEQ ID NO: 10) |
| mdm2-P20L-QC1: | 5′-CCACCTCACAGATTCTAGCTTCGGAACAAGA-3′ |
| | (SEQ ID NO: 11) |
| mdm2-P20L-QC2: | 5′-TCTTGTTCCGAAGCTAGAATCTGTGAGGTGG-3′ |
| | (SEQ ID NO: 12) |
| mdm2-Q24R-QC1: | 5′-TTCCAGCTTCGGAACGAGAGACCCTGGTTAG-3′ |
| | (SEQ ID NO: 13) |
| mdm2-Q24R-QC2: | 5′-CTAACCAGGGTCTCTCGTTCCGAAGCTGGAA-3′ |
| | (SEQ ID NO: 14) |
| HDMM62A-1: | 5′-CTTGGCCAGTATATTGCGACTAAACGATTATATG-3′ |
| | (SEQ ID NO: 15) |
| HDMM62A-2: | 5′-CATATAATCGTTTAGTCGCAATATACTGGCCAAG-3′ |
| | (SEQ ID NO: 16) |
| mdm2-M62V-QC1: | 5′-CTTGGCCAGTATATTGTGACTAAACGATTAT-3′ |
| | (SEQ ID NO: 17) |
| mdm2-M62V-QC2: | 5′-ATAATCGTTTAGTCACAATATACTGGCCAAG-3′ |
| | (SEQ ID NO: 18) |
| mdm2-V280A-QC1: | 5′-TATATCAAGTTACTGCGTATCAGGCAGGGGA-3′ |
| | (SEQ ID NO: 19) |
| mdm2-V280A-QC2: | 5′-TCCCCTGCCTGATACGCAGTAACTTGATATA-3′ |
| | (SEQ ID NO: 20) |
| mdm2-G443D-QC1: | 5′-GTGTGATTTGTCAAGATCGACCTAAAAATGG-3′ |
| | (SEQ ID NO: 21) |
| mdm2-G443D-QC2: | 5′-CCATTTTTAGGTCGATCTTGACAAATCACAC-3′ |
| | (SEQ ID NO: 22) |
| 2CONART-F: | 5′-GGCATGTCCGCTGAGTC-3′ |
| | (SEQ ID NO: 23) |
| WpetR1: | 5′-TAATTTCGCGGGATCGAGATCT-3′ |
| | (SEQ ID NO: 24) |

### Vector and HDM2 Library Construction

Inverse PCR was carried out on vector PET22b with primers petF3conA-Rlink and petF4conA-Flink and the PCR products were ligated intramolecularly to construct 2ConA-PET22b. The same inverse PCR was carried out on HDM2-PET22b to construct 2ConA-HDM2-PET22b.

Error-prone PCR [54] was carried out on HDM2-PET22b using primers petF2 and petR and mutant genes re-amplified with Hdm2-Nde1 and Hdm2-HA-BamH1. The library was ligated into 2ConA-PET22b via Nde1/BamH1 sites and re-amplified with petF2 and petR to make library amplicons with T7 promoter and ribosome binding site required for in vitro transcription-translation (IVT), as well as the 2ConA RE site located before the T7 promoter site. 2ConA-HDM2-PET22b, HDM2-PET22b and p53-PET22b were also amplified with petF2 and petR for IVT of wild-type HDM2 and p53.

Nutlin-resistant parental clones obtained from the selection were amplified with petF2 and petR to create amplicons for secondary assays. Three parental clones (5-3, 5-9 and 5-14) were also amplified with INF-Hdm2-cmvF and INF-HA-cmvRcor for cloning by infusion (Clontech) into the pCMV vector expression in cells.

Single mutant HDM2 clones were generated by Quickchange mutagenesis (Stratagene) of 2ConA-HDM2-PET22b using primers mdm2-T16A-QC1 and mdm2-T16A-QC2, mdm2-P20L-QC1 and mdm2-P20L-QC2, mdm2-Q24R-QC1 and mdm2-Q24R-QC2, HDMM62A-1 and HDMM62A-2, mdm2-M62V-QC1 and mdm2-M62V-QC2, mdm2-V280A-QC1 and mdm2-V280A-QC2, mdm2-G443D-QC1 and mdm2-G443D-QC2 to create 2ConA-HDM2-T16A-pET22b, 2ConA-HDM2-P20L-pET22b, 2ConA-HDM2-Q24R-pET22b, 2ConA-HDM2-M62A-12 pET22b, 2ConA-HDM2-M62V-pET22b, 2ConA-HDM2-V280A-pET22b and 2ConA-HDM2-G442D-pET22b, respectively. The same primers were used to introduce mutations into the parental pCMV-HDM2 mammalian expression construct.

### In Vitro Selection of HDM2 Variants Resistant to Nutlin

IVC reactions consisting of 0.5 μM ZnCl₂, 8 ng p53 (1.6 ng and 0.8 ng in subsequent rounds), 5 ng library amplicons (ing and 0.5 ng in subsequent rounds) in a total volume of 50 μL PURExpress® in vitro protein synthesis solution (New England Biolabs) were assembled on ice and emulsified in 450 μL in vitro compartmentalization (IVC) oil comprising 95% (v/v) mineral oil, 4.5% (v/v) Span-80 and 0.5% (v/v) Tween-80 as previously described [17]. After incubation at 37° C., the reactions were centrifuged at 8000 rpm for 10 mins to separate the aqueous and oil phase. The oil phase was removed and 50 uL TNTB buffer (0.1M Tris pH 7.4, 0.15M NaCl, 0.05% Tween-20, 0.5% BSA) was added to the pellet of aqueous phase compartments. The compartments were disrupted by six rounds of hexane extraction and the aqueous phase incubated with anti-HA antibody-coated protein G beads (Invitrogen) at 4° C. with rotation. The beads were washed thrice with PBST-0.1% BSA, and thrice with PEST. The beads were resuspended in 20 μl water and the protein-protein-DNA complexes eluted by incubation at 95° C. for 5 mins. The eluates were amplified with Hdm2-Nde1 and Hdm2-HA-BamH1 and products cloned back into 2ConA-PET22b via Nde1/BamH1 sites and re-amplified with petF2 and petR for the next round of selection.

### Secondary Co-Immunoprecipitation Assay and Western Blot Analysis

Protein G beads were incubated with anti-HA (1 μg per 10 μL beads) for 1 hour in PEST-3% BSA and subsequently washed twice in PBST-0.1% BSA. IVT-expressed protein was incubated with the beads on a rotator for 30 mins. Nutlin was added at required concentrations and incubation carried out for 30 mins. IVT-containing secondary protein was added to the mixture and incubation allowed for 1 hour. Beads were finally washed thrice in PBST-0.1% BSA and thrice with PBS, and bound proteins eluted by resuspension in 20 μL SDS-PAGE loading buffer and incubation at 95° C. for 5 minutes. Where required, blank IVT extract (no template DNA added) was used as control. The eluates were subjected to electrophoresis, transferred to nitrocellulose membranes and probed for p53 with horseradish peroxidise conjugated DO1 antibody (Santa Cruz) or for HDM2 with anti-HA antibody followed by rabbit anti-mouse (Dakocytomation).

### Proof-of-Principle DNA Binding Assay and Real-Time PCR

Protein G beads were incubated with anti-HA (1 μg per 5 μL beads) for 1 hour in PBST-3% BSA and subsequently washed twice in PBST-0.1% BSA. IVT-expressed HDM2 (with either HDM2 or HDM2 2ConA as template DNA) was incubated with the beads on a rotator for mins. Nutlin was added at required concentrations and incubation carried out for 1 hour. IVT-expressed p53 was added to the mixture and incubation allowed for 1 hour. Beads were finally washed thrice in PBST-0.1% BSA and thrice with PBS, and bound DNA eluted by resuspension in 20 μL nuclease-free water and incubation at 95° C. for 5 minutes. Real-time PCR quantifications of the eluates were performed using 250 nM each of primers 2CONART-F and WpetR1 using iQTM SYBR® Green Supermix (Bio-Rad Laboratories) and quantified via CFX96 Real-Time System CCD camera (Bio-Rad Laboratories). Data was interpreted as fold differences (calculated based on cycle threshold differences) over non-specific DNA binding control (HDM2 DNA).

### Cell Culture and Reporter Assay

DKO (p53/HDM2 null) cells were maintained in Dulbecco's modified Eagle's medium (DMEM) with 10% foetal calf serum (FCS) and 1% penicillin/streptomycin. The cells were seeded at 1.0×10⁵ cells/well in 6-well plates, 24 hours prior to transfection. Cells were co-transfected with parental or individual Nutlin-resistant HDM2 plasmid, p53-pcDNA plasmid, LacZ reporter plasmid and luciferase transfection efficiency plasmid using TurboFect transfection reagent (Thermo Scientific) according to the manufacturer's instructions. Nutlin was added to selected wells at required concentrations 4.5 hours post-transfection. In all cases, the total amount of plasmid DNA transfected per well was equilibrated by addition of the parental vector pcDNA3.1a(+).

### β-Galactosidase Assay and Western Blot Analysis

DKO cells were harvested 24 hours after transfection and 3-galactosidase activities were assessed using the Dual-light System (Applied Biosystems) according to the manufacturer's protocol. The β-galactosidase activity was normalized with luciferase activity for each sample. To check for expression levels of relevant proteins via Western blot, 2.5 μg of the cell lysates were subjected to electrophoresis, transferred to nitrocellulose membranes and probed for p53 with horseradish peroxidise conjugated DO1 antibody, for HDM2 and actin with anti-HA antibody and AC15 antibody respectively followed by rabbit anti-mouse.

### F2H Co-Localization Assay

The Fluorescent 2-Hybrid (F2H) assay is an intracellular, direct, fully reversible protein-protein interaction assay. This microscopy-assisted assay consists of two components, a bait and a prey protein. Here, the bait is a fusion of p53 (amino acid 1-81) with a lac repressor binding domain (LacI) and GFP. The prey is a fusion of RFP with either N-terminal domain of wild-type HDM2, mutants Q24R or M62A (amino acids 7-134). These two plasmids are co-transfected in a specific transgenic BHK cell line containing an array of lac operator repeats, the F2H interaction platform. The bait protein is then captured at this interaction platform and forms a bright green spot in the cell nucleus [27]. Upon interaction the prey protein co-localizes to the same nuclear spot. Compounds, which disrupt the protein-protein interaction (here Nutlin) are titrated onto the cells and the declined percentage of co-localization is measured using imaging techniques.

For testing the nutlin-resistance of the mutant HDM2s, BHK cells were co-transfected with the bait p53 and different prey HDM2 plasmids overnight in 96 multiwell plates (μClear Greiner Bio-One, Germany) using the Lipofectamine 2000 (Life Technologies) reverse transfection protocol according to the manufacturer's instructions with 0.2 μg DNA and 0.4 μl Lipofectamine 2000 per well. Cells were incubated with a dilution series of 50 μM, 10 μM, 2 μM, 1 μM, 0.5 μM, 0.25 μM and 0.13 μM Nutlin for 1 hour at 37° C., 5% CO₂.

Interaction (%) was determined as the ratio of cells showing co-localization of fluorescent signals at the nuclear spot to the total number of evaluated cells. For automated image acquisition, an INCell Analyzer 1000 with a 20× objective (GE Healthcare) was used. Automated image segmentation and analysis was performed with the corresponding INCell Workstation 3.6 software. At least 100 co-transfected cells were analyzed per well. Titrations were carried out independently three to five times.

### Molecular Dynamics Simulations

### Interactions Between the N Terminal Domain of HDM2 and the N Terminal Domain of p53 or Nutlin

To model the interactions of the N terminal domain of HDM2 with p53 and Nutlin, the crystal structures of the HDM2-p53 complex [19] (PDB code 1YCR, resolved at 2.6 Å) and the HDM2-Nutlin complex [38] (PDB code 1RV1, resolved at 2.3 Å) were used. The N terminus of HDM2 was extended from residue 25 (as in 1YCR) by grafting residues 19-24 from 4ERF [46] (resolved at 2.0 Å) on to 1YCR. This yielded a final HDM2 with residues 19-109 of human HDM2; the p53 segment from residues 17-29 as found in 1YCR was used and Nutlin from 1RV1 was used. The N- and C-termini were capped with acetyl (ACE) and N-methyl (NME) respectively to keep them neutral. Molecular dynamics simulations were performed with the SANDER module of the AMBER11 [55] package employing the all-atom Cornell force field [56]. Nutlin parameters were built using antechamber [57]. All systems were prepared as described before [16] and simulated for 100 ns at constant temperature (300K) and pressure (1 atm) and structures were stored every fps. The free energies of binding (ΔG_{bind}) of the p53 and Nutlin to MDM2 were computed and visualizations were carried out as described earlier [16].

### Interactions Between the Acidic Domain of Mdm2 and the DNA Binding Domain (DBD) of p53

The acidic domain of MDM2 is known to be unstructured [37], and hence molecular dynamics in implicit solvent (AMBER molecular modeling package) [55] was used to model a 24-residue peptide in the acidic domain (residues 259-282 of MDM2) starting from an extended chain. These structures were then used as input into the program HADDOCK [58], which docks molecules based on geomteric restraints between two sets of “active” residues. The “active” residues are chosen based on available experimental data. For MDM2, residues (2, 4, 12, 13, 14, 15, 16, 18, 19 or corresponding residue numbers in MDM2: 260, 262, 270, 271, 272, 273, 274, 276, 277) were selected based on experimental data [8, 10] as active while for the p53 protein monomer of the core domain in the absence of DNA (PDB id: 2OCJa) was used, and the active residues considered to be important for peptide binding [66] were (residues 114, 115, 117, 118, 279, 280, 282, 283, 286, 248) chosen. These restraints were then processed and structures optimized by HADDOCK and were then refined using Rosetta's FlexPepDocking protocol[59] to obtain more diverse structures that are still consistent with the experimental constraints. The same procedure was repeated for the V280A mutant.

### Results

### In Vitro Selection of Nutlin-Resistant HDM2

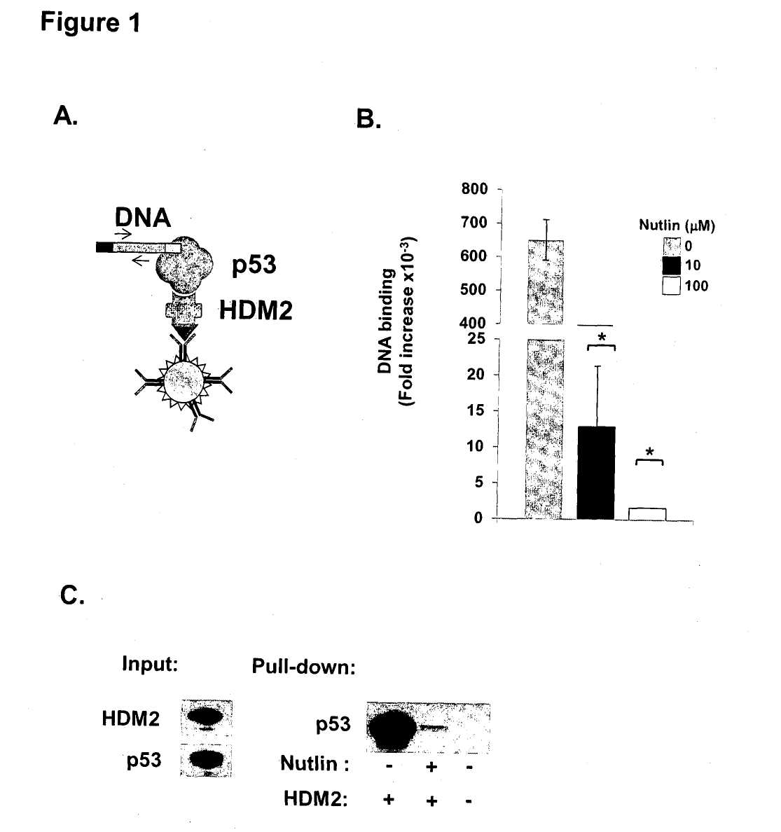

A previous in vitro compartmentalization (IVC) selection for p53 variants with altered binding specificities linked genotype with phenotype by p53 binding back to the gene encoding it via an appended p53 DNA response element (RE) [17]. To enable selection of HDM2 variants capable of binding p53 in the presence of Nutlin, it was first determined whether an HDM2-p53-DNA complex is able to form in vitro. HA-tagged HDM2 was expressed in vitro from a DNA template to which two copies of the p53 CONA response element [18] were appended. Magnetic beads coated with anti-HA antibody were added to the in vitro reaction to capture the HDM2 protein, following which p53 protein (also expressed in vitro) was added. After incubation, the beads were washed and DNA captured on the beads quantified by real-time PCR (FIG. 1A). A control reaction was also carried out where the DNA template encoding HDM2 did not have the 2CONA RE appended. The results in FIG. 1B show that DNA is only captured on the beads when the 2CONA RE is present, indicating the formation of an HDM2-p53-DNA complex. Importantly, addition of Nutlin resulted in a clear dose-dependent reduction in the amount of 2CONA-appended DNA pulled down (˜383 fold reduction at 100 μM), indicating disruption of the p53-HDM2 interaction in vitro. Disruption was also observed in a pull-down assay measuring p53 bound to immobilised HDM2 by Western blot (FIG. 1C).

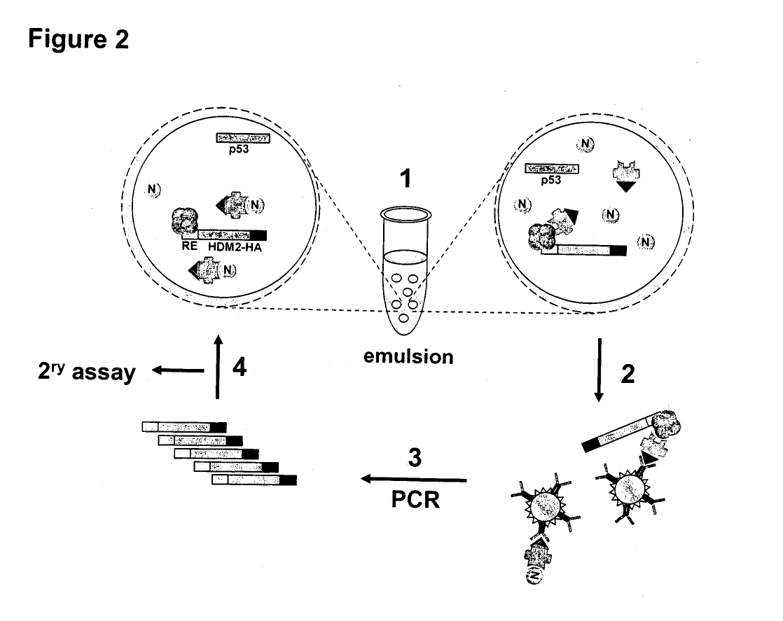

Based on these results, a library of randomly mutated HDM2 genes was created and a selection for Nutlin-resistance by IVC was carried out. FIG. 2 depicts the selection protocol, wherein variant HDM2 expression constructs tagged with the CONA RE, along with p53 expression construct and Nutlin are dispersed into the aqueous compartments of the water-in-oil emulsion. Within each compartment protein expression occurs, and in the presence of Nutlin, the HDM2-p53-DNA complex is not expected to form if Nutlin binds HDM2 (left bubble), but will form if the variant HDM2 is resistant to Nutlin inhibition (right bubble). After formation of complexes, the emulsion is broken and complexes are captured using anti-HA coated magnetic beads. The genes encoding Nutlin-resistant HDM2 variants are then amplified by PCR prior to further rounds of selection and/or secondary characterisation.

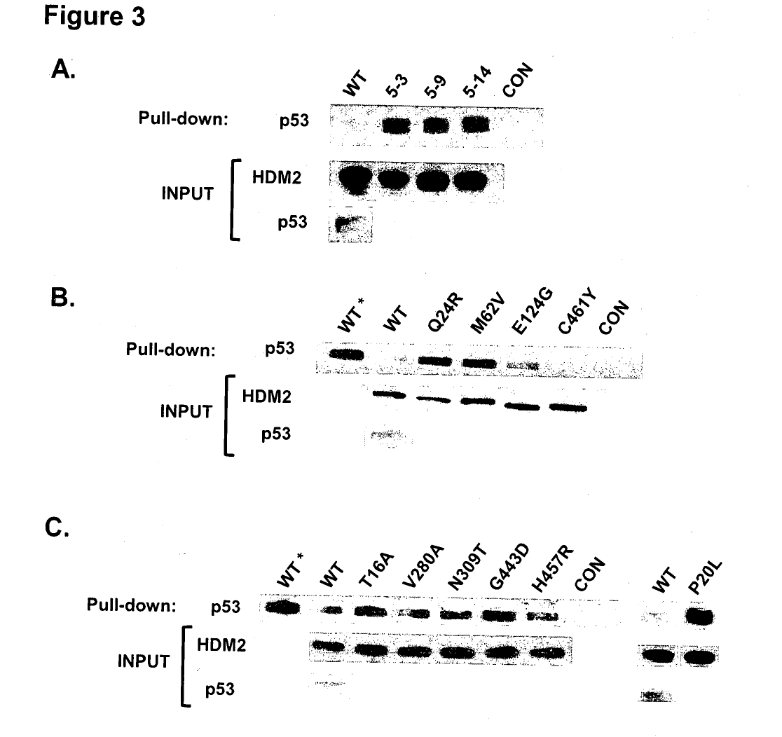

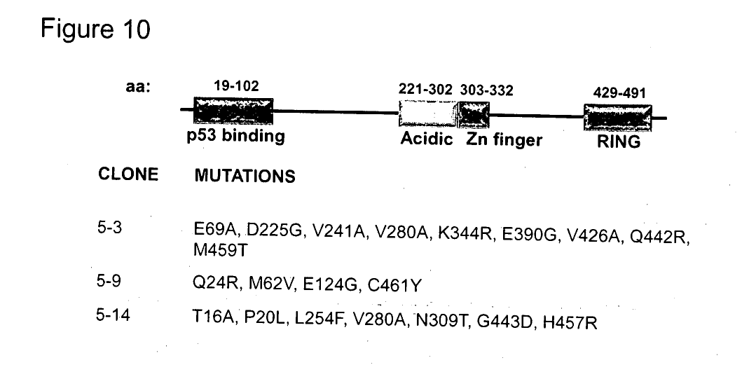

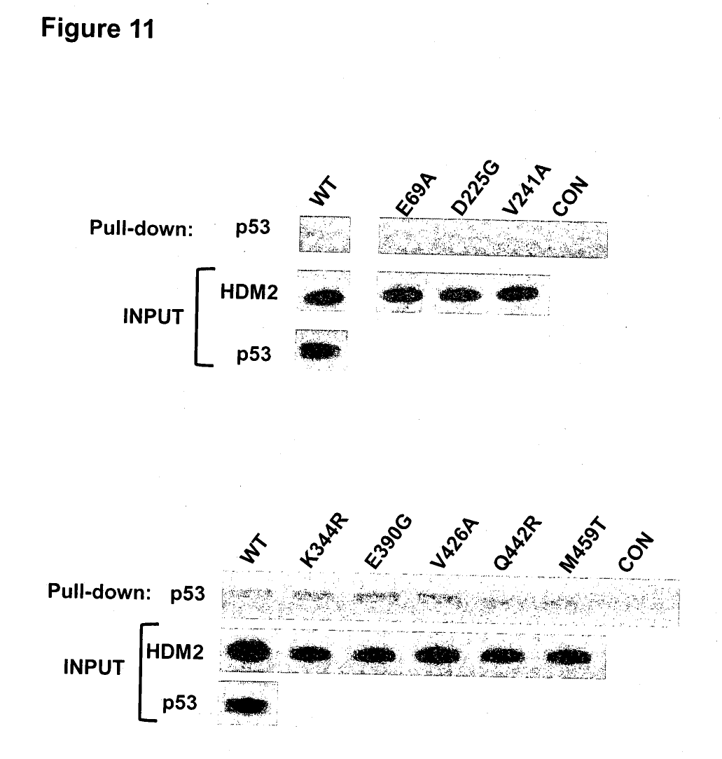

After 5 rounds of selection using the above protocol, 15 clones were selected and analysed in a secondary pull-down assay. Of these, 3 clones (Clones 5.3, 5.9, 5.14) showed significantly more binding to p53 in the presence of Nutlin compared to wild-type HDM2 (FIG. 3A). Sequence analysis indicated several mutations in the N-terminal p53/Nutlin binding domain (amino acids 19-102) [19,20]. Additionally, mutations were seen in the central acidic (amino acids 221-302) [21,22], zinc-finger (amino acids 303-332)[23] and RING (amino acids 429-491) [24] domains (FIG. 10). Investigation of the individual contribution of each mutation indicated that T16A, P20L, M62V, E124G (N-terminal domain), V280A (acidic domain), N309T (zinc finger domain) and G443D (RING domain) in isolation conferred Nutlin resistance (FIGS. 3B,3C). Apart for the V280A mutation in HDM2-5.3 (also present in HDM2-5.14), the remaining mutations in this clone did not display significant resistance when assayed in isolation (FIG. 11). It is possible that these mutations are epistatic for the resistance phenotype.

### In Vitro Selectants Display Nutlin-Resistant Phenotype in Function Cell Assay

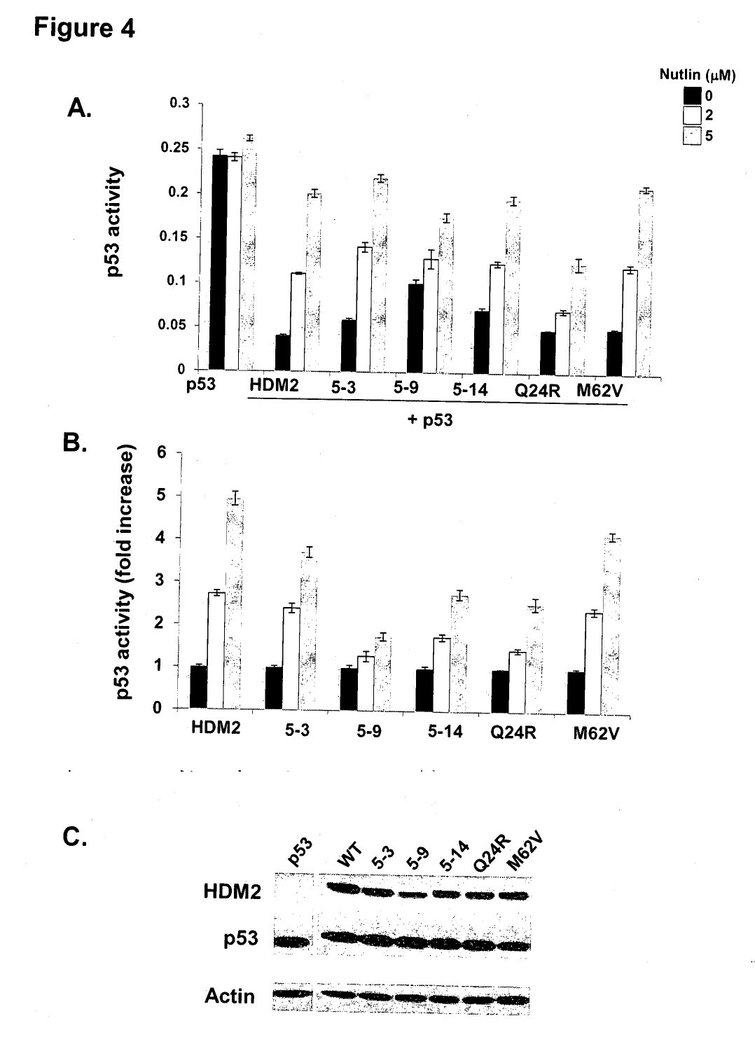

The parental selectants and the single HDM2 mutants Q24R and M62V were next analysed in a functional assay measuring p53 activity in the H1299 cell line (FIG. 4A). Plasmids encoding HDM2 (wild-type or selectants) and p53 were transfected along with a p53 transactivation reporter construct. In the presence of Nutlin, inhibition by HDM2 was attenuated, with p53 activity being restored up to 76% of that observed in the absence of HDM2 co-transfection (5 μM Nutlin). HDM2 Q24R showed an appreciable Nutlin-resistant phenotype, with p53 activity only being restored to 47% (5 μM Nutlin). Whilst the parental clones did not show a net resistance phenotype, Nutlin was clearly less effective on these mutants when p53 activity was compared to the basal value of inhibition in the absence of drug (FIG. 4B). This value was elevated for all the parental clones, most likely due to their reduced expression levels compared to wild-type HDM2 (FIG. 4C), particularly selectant 5.9.

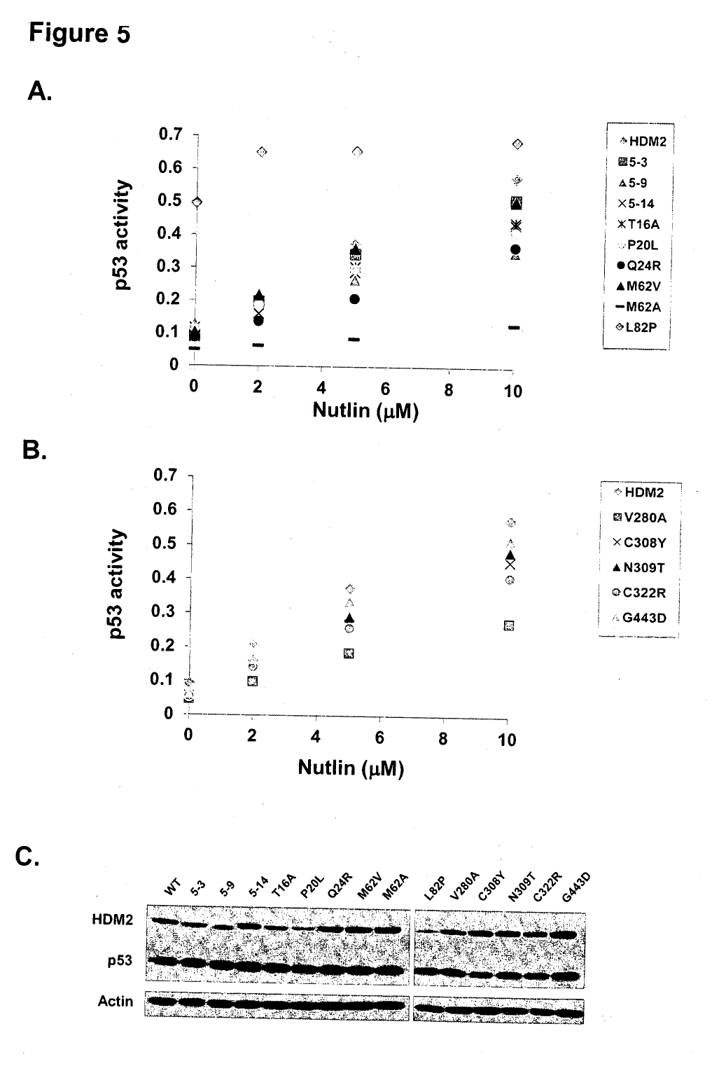

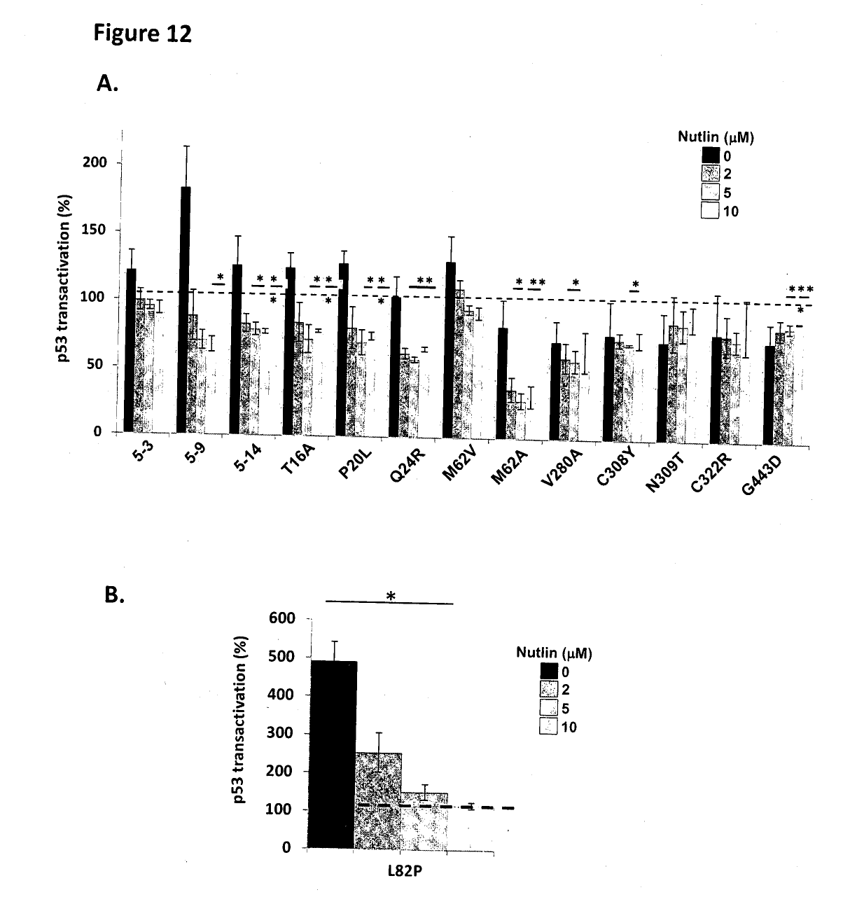

As these results were possibly affected by the presence of endogenous HDM2 in H1299 cells, the assay was repeated in the p53/HDM2-null DKO cell line [25] (FIGS. 5, 12). In this cell line, the parental HDM2-5.9 and 5.14 selectants displayed a resistance phenotype at all Nutlin doses tested, as shown by the reduced p53 activation compared to HDM2. Analysis of individual mutations indicated Q24R, P20L, and T16A in the N-terminal domain to elicit moderate resistance phenotypes, all showing between 50-80% restoration of activity seen with wild-type HDM2. M62V did not display any significant resistance. Sequence analysis of round 5 selectants showed the mutation L82P to occur in two independent clones. This point mutant was therefore investigated, and despite the higher baseline value in the absence of Nutlin (most likely due to reduced expression level, see FIG. 5C), it was highly Nutlin resistant, with very little restoration of p53 activity at the highest dose (˜1.5-fold increase compared to ˜6.4-fold increase seen for wild-type HDM2). The M62A mutant was also included, previously shown only by in vitro pull-down to be Nutlin resistant [26]. This mutant showed strong resistance in this assay, with p53 activity only being restored to ˜30% of that seen with HDM2. Within the acidic domain, V280A showed ˜55% activity of wild-type. The N309T mutant in the zinc finger domain showed slight resistance (˜88% activity of wild-type). However, its proximity to C308, shown to be mutated in non-Nutlin treated cancer [12] led us to test the clinically observed C308Y mutant and this showed moderate resistance (˜70% activity of wild-type). The C322R mutation, also in the zinc finger domain also showed moderate resistance (˜73% activity of wild-type). The G443D mutant in the RING domain showed slight resistance (˜85% restoration).

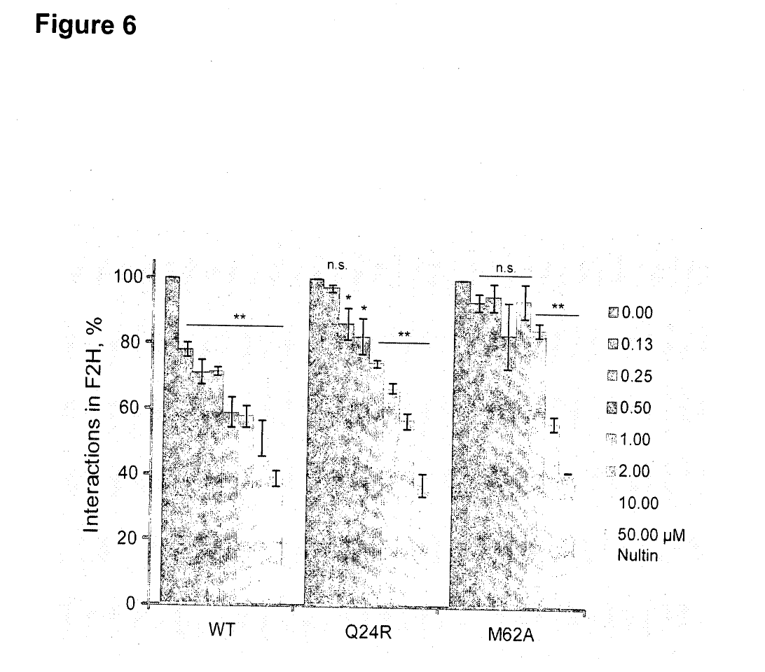

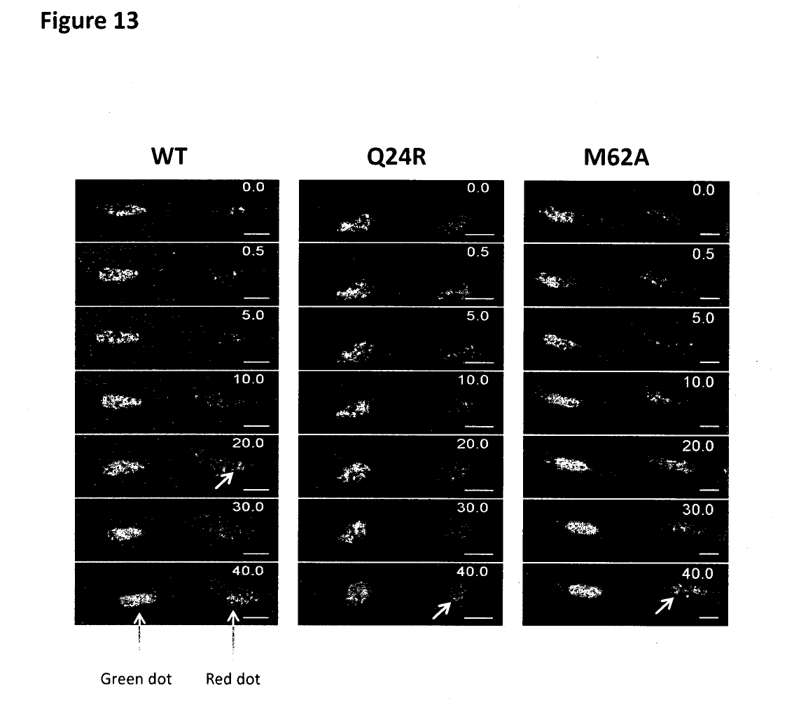

The direct cellular binding of the HDM2 wild-type, Q24R and M62A N-terminal domains to p53 were further characterized in the Fluorescent 2-Hybrid (F2H) assay [27]. The F2H assay differs from the DKO reporter assay in that it does not measure reactivation of a reporter gene but the precise interaction to be disrupted. The assay visualizes the interaction of RFP-tagged HDM2 (amino acids 7-134) with GFP-tagged p53 (amino acids 1-81) at a defined nuclear F2H interaction platform, in specific BHK cells. The addition of Nutlin results in a dissociation of the complex, which can be imaged and quantified. Compared to the wild-type HDM2-p53 interaction, addition of Nutlin resulted in reduced dissociation of mutant N-terminal domains from p53, indicating Nutlin resistance (FIG. 6). For wild-type HDM2 a highly significant reduction of interactions was measured at 0.13 μM Nutlin. For the two mutants Q24R and M62A, no significant reduction was detectable until addition of at least ten times higher concentrations of Nutlin (1 and 2 μM respectively). The Q24A interaction with p53 was disrupted less significantly in the range of 0.25-1 μM Nutlin. M62A clearly showed a stronger phenotype than Q24R, which is in accordance with the reporter assay in DKO cells. Furthermore, time-lapse analysis indicated enhanced persistence of mutant HDM2-p53 complexes compared to wild-type after Nutlin challenge (1 μM). The wild-type complex was not visible after 20 minutes, whilst the Q24R complex lasted for 40 minutes. The M62A complex was still visible after one hour (FIG. 13).

### Discussion

IVC was used to select for Nutlin-resistant variants from a large repertoire (˜10⁹) of randomly mutated HDM2 genes. It is desirable to increase selection pressure during rounds of directed evolution. However, the present study was restricted to some extent by the low solubility limit of Nutlin [28] and its strong hydrophobicity, which most likely led to much of it partitioning into the oil phase of the emulsion. Despite this, enough selection pressure was applied to yield several clones harbouring multiple mutations which showed the desired phenotype. As acquired drug resistance can arise through point mutation [29,30,31], the mutations were analysed in isolation, and several of these displayed the Nutlin-resistant phenotype both in vitro and ex vivo. Some discrepancy was however observed between the two assay formats. For example, HDM2-5.3 showed appreciable binding to p53 in the presence of Nutlin in the in vitro pull-down assay, but displayed a mild phenotype in the ex vivo functional assay. The first assay measures binding of HDM2 to p53, whilst the second is the aggregate readout for inhibition of p53 activity arising from the binding, inhibition of transactivation, and E3 ligase activities of HDM2. Hence, mutations ancillary to V280A in selectant 5.3 (which confers Nutlin resistance in isolation) likely impact negatively on the latter two activities in the ex vivo assay. The V280A mutation is also present in selectant 5.14 which shows essentially the same phenotype in both assays, indicating context-dependency. Overall, the assumption that in vitro binding can be used as a proxy to measure HDM2 function in the cell-based assay is validated, through selection of HDM2 variants 5.9 and 5.14 which behave similarly in both assays.

Residues 16-24 in the p53 binding domain of apo HDM2 comprise a flexible lid region shown to behave as a weak pseudo-substrate in the absence of p53 binding [32,33]. NMR studies indicate that whilst the lid predominantly adopts the “open” conformation when p53 is bound, Nutlin-binding is compatible with both the “open” and “closed” lid-binding states [34]. Hence, the mutations T16A, P20L and Q24R may further weaken this intra-molecular interaction to selectively increase the interaction with p53. In support of this model, biochemical studies have shown the phosphomimetic mutation S17D in the lid to stabilize the HDM2-p53 interaction [35,36]. A similar model, suggested by molecular simulations of the complexes of HDM2 (with lid) and p53/Nutlin indicates that P20 makes weak interactions with the hydrophobic side chains of L26 and P27 of p53. Mutation to the more hydrophobic leucine is predicted to selectively enhance these interactions which are absent when Nutlin is the ligand.

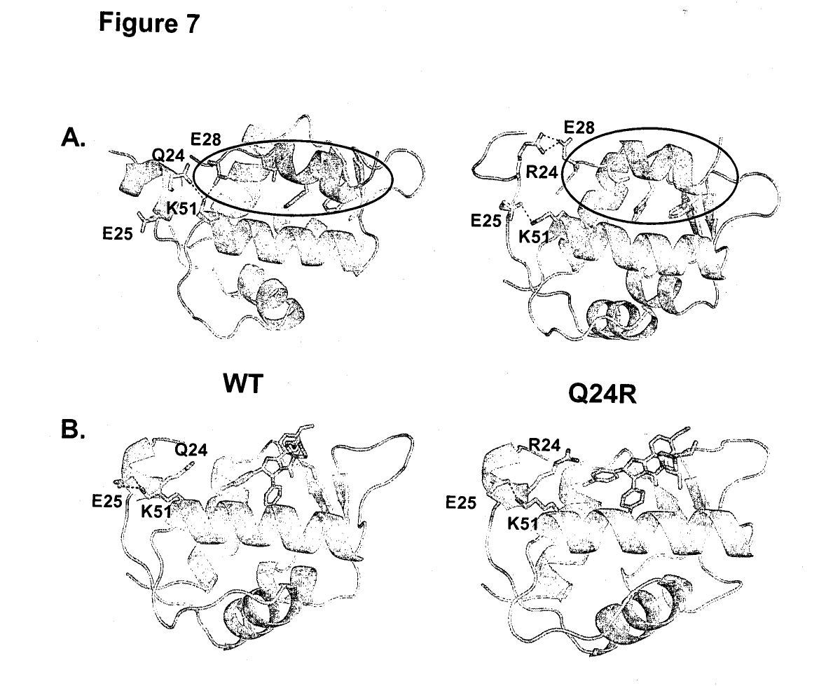

Studies have shown that K51 of HDM2 interacts with E28 of p53 [37]. Simulations indicate that the Q24R mutation leads to the development of a cationic potential in the region of R24. This results in repulsion of K51 which in turn stabilizes anionic E28 of p53 through a charge-charge interaction and enhances the affinity of p53 for HDM2 (FIG. 7A). No such interaction is possible with Nutlin, thus R24 remains solvent exposed (FIG. 73). The energetics of binding further show this trend (FIG. 12), and this mutant appears to confer resistance by stabilizing p53 binding without affecting Nutlin binding. A similar mechanism has been described for a point mutant of the EFGR kinase which causes resistance to the ATP-analogue gefitinib by increasing affinity for ATP [30].

The L82P mutation in the N-terminal domain conferred significant Nutlin-resistance. L82 lies within the al′ helix forming part of the floor of the hydrophobic p53-binding pocket. The lower expression level of this mutant suggests that substitution to a less hydrophobic amino acid destabilizes the overall N-terminal domain fold. Conformational changes associated with this destabilization could preferentially abrogate Nutlin binding as it makes fewer contacts with HMD2 than p53 [38,19]. Additionally, p53 binding could preferentially stabilize this mutant by shielding otherwise solvent-exposed hydrophobic residues.

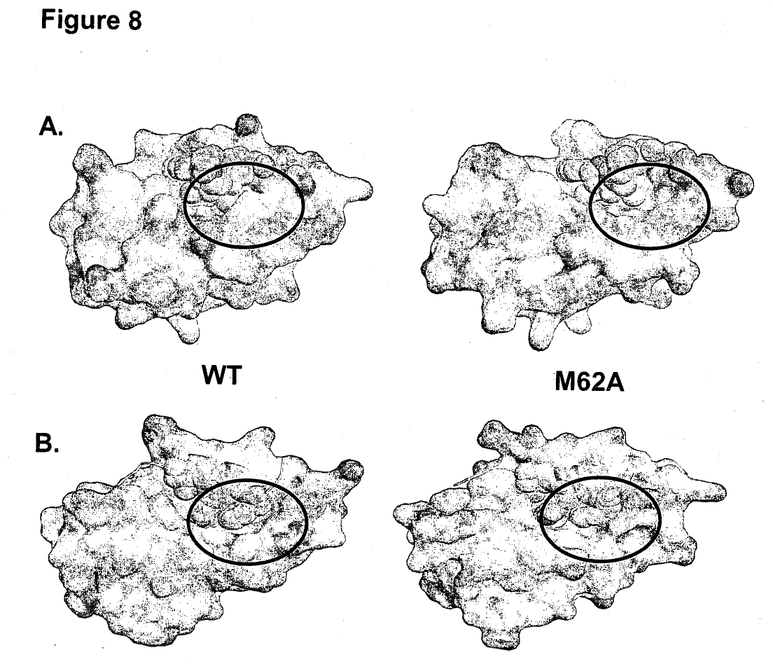

Selection of the M62V mutation was of particular interest as it was previously shown that M62A confers Nutlin resistance in vitro [26]. The amino acid M62 is an essential part of the subpocket accommodating F19 of p53 in the HDM2-p53 interaction [19], and small molecules such as Nutlin designed to mimic the three key interactions (F19, W23 and L26) of p53, will also interact with this subpocket. M62 makes direct contacts with both p53 and Nutlin in their respective crystal structures [38,19]. However, the mutation M62A causes the loss of a significant fraction of packing interactions with Nutlin, thus selectively destabilizing its binding with less impact on p53 which makes contacts with HDM2 over an extended surface (FIG. 8). Again, the energetics of the interactions shows that binding of Nutlin is destabilized (FIG. 12) while that of p53 is marginally affected, thus providing a mechanism for the observed resistance. Mutation of methionine to alanine would require mutagenesis of two adjacent nucleotides (AT to GC), which is unlikely to occur using the error-prone PCR mutagenesis employed in this study. However, mutation to valine, one of the more similar amino acids to alanine, required only a single base change. The weaker ex vivo phenotype of M62V compared to M62A suggests that mutation to valine impacts negatively on post p53-binding events described above.

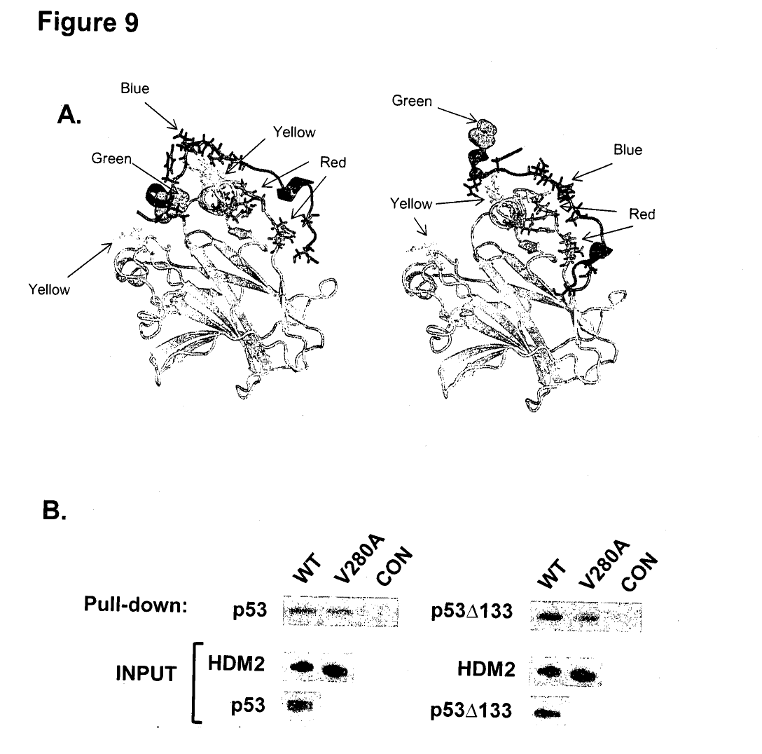

The central acidic domain of HDM2 contains a secondary binding site that interacts with the p53 DNA binding domain [37,39]. Mechanistically, it could be expected that mutations in this region that increase the secondary interaction might be selected to counteract Nutlin-induced loss of the primary interaction site. However, for the V280A mutation which lies within the secondary binding site, in silico prediction suggested the converse, and this was subsequently verified by in vitro pulldown (FIG. 9). This points to an allosteric mechanism, as previously shown for mutations in the HDM2 C-terminal RING domain, that impact on Nutlin binding [40]. Such a mechanism could further account for the other mutations identified in this study that lie outside the HDM2 p53-binding domain. The central regions of HDM2 additionally interact with negative regulators including ARF and RPL11. Notably, cancer-associated mutations including C308Y in the central zinc finger domain have been described (in non-Nutlin treated individuals) which disrupt interaction with RP11, a potent negative regulator of HDM2 [41,42,43,44,45]. Led by the in vitro selection of the N309T zinc finger domain mutation, the C308Y mutation was investigated, and this conferred Nutlin-resistance. Hence, there is precedence for future clinical resistance arising through mutations in the central acidic and zinc finger domains which concurrently inhibit binding of both Nutlin and regulatory proteins.

The present data indicates that resistance to Nutlin can arise through several mechanisms. In the case of the N-terminal domain HDM2 mutants, these are predicted to either selectively reduce affinity for Nutlin (M62A, M62V, L82P), increase affinity for p53 (P20L, Q24R), or influence lid dynamics (T16A, P20L, Q24R). These effects can possibly be overcome by designing small molecule derivatives capable of forming additional contacts with the HDM2 binding pocket. Recently described examples include a series of piperidinones, which in addition to the three core p53-mimetic interactions, form additional Van der Waals, pie-stacking and electrostatic interactions with HDM2 [46]. Alternatively, stapled-peptide derivatives of the p53 motif that interact with HDM2 may prove more recalcitrant to mutation by virtue of the increased interaction footprint [47,48]. The absence of structural data for full-length HDM2 makes it difficult to understand probable allosteric effects of mutations outside the N-terminal domain that impact on Nutlin binding (V280A, C308Y, N309T, C322R, G443D). However, C-terminal RING domain mutants have been described which increase the affinity of the HDM2-p53 interaction [40]. Therefore, as with N-terminal domain mutations that increase p53-binding, the use of small molecules and stapled peptides with increased binding footprints may offset allosterically induced structural variation and compete more efficiently with p53 for binding.

The experimental approach to anticipating cancer drug resistance has most commonly involved the treatment of drug-sensitive cell lines, followed by analysis of resistant subpopulations [49]. Non-targeted in vitro mutagenesis of a cell line, followed by treatment and selection for resistance has also been described [50]. Target-based mutagenesis approaches, wherein complementation by a mutated protein enables survival of an otherwise drug-sensitive cell line, have correctly anticipated drug resistance [51,52]. However, a major disadvantage is the relatively small library of variants that can be sampled due to inherent technical limitations (˜10⁶), and the possibility of off-target drug toxicity at higher doses limiting selection pressure. By comparison, IVC readily enables interrogation of up to 10¹⁰ variants [53] and generally allows for application of stringent selection pressures (although in this particular case the physicochemical properties of Nutlin were limiting). Being completely in vitro, IVC may not be suitable where the function of target proteins requires post-translational modification, and for certain targets it may not be trivial to devise a selection strategy. However, where in vitro selection is possible, a robust approach to modeling drug resistance could entail primary use of IVC to sample a large pool of diversity for mutation hotspots. Smaller, focused libraries covering these regions could then be generated, and these further analysed in cell-based complementation assays.

The present study anticipates a broad spectrum of novel resistance mutations in HDM2 which may arise in the clinic. Mechanistic insights gleaned from this mutation will aid in future drug design and furthers the current understanding of the complex p53-HDM2 interaction. In this regard, it is hypothesized that these mutations could be overcome through iterative structure guided chemical modification of Nutlin, or the use of antagonists with a larger interaction footprint.

### Example 2

### Inhibition of Nutlin-Resistant HDM2 Mutants by Stapled Peptides

Stapled peptides are a relatively new class of macrocyclic compounds with promising drug-like properties [60]. The introduction of a covalent linkage bridging adjacent turns of an alpha helical peptide (the “staple”), can pre-stabilize the conformer(s) preferentially adopted when it binds a target protein. Stapling increases affinity by reducing the entropic cost of binding, imparts proteolytic stability/increased in vivo half-life, and in certain cases permits adjunct-free cellular uptake [61-63]. Stapled peptide analogues of Nutlin that target the N-terminal domain of HDM2 have been described [47,64], and these mimic the contiguous stretch of p53 (residues 18 to 26) that bind the N-terminal hydrophobic pocket in an α-helical conformation [19,65,66]. As these stapled peptides form significantly increased contacts with HDM2 compared to Nutlin [48,67], they may prove recalcitrant to mutations that reduce Nutlin efficacy.

The present data indicates this to be the case, as shown both experimentally and further rationalized by molecular dynamics simulations. The ability of stapled peptides to form comparatively more contacts with target proteins may therefore prove detrimental to the emergence of acquired resistance should this drug-class enter the clinic.

### Materials and Methods

Unless otherwise specified, all oligonucleotides used in this work were from 1st Base (Singapore), restriction enzymes from NEB and chemical reagents from Sigma. Nutlin-3A was from Calbiochem. The stapled peptides PM2, PM2CON and MO11 (>90% purity) were from AnaSpec (USA).

Primers

| | | |
|---|---|---|
| 1) | HDM2-P20L-QC1: | 5′-CCACCTCACAGATTCTAGCTTCGGAACAAGA-3′ |
| | | (SEQ ID NO: 25) |
| 2) | HDM2-P20L-QC2: | 5′-TCTTGTTCCGAAGCTAGAATCTGTGAGGTGG-3′ |
| | | (SEQ ID NO: 26) |
| 3) | HDM2-Q24R-QC1: | 5′-TTCCAGCTTCGGAACGAGAGACCCTGGTTAG-3′ |
| | | (SEQ ID NO: 27) |
| 4) | HDM2-Q24R-QC2: | 5′-CTAACCAGGGTCTCTCGTTCCGAAGCTGGAA-3′ |
| | | (SEQ ID NO: 28) |
| 5) | HDM2-M62A-1: | 5′-CTTGGCCAGTATATTGCGACTAAACGATTATATG-3′ |
| | | (SEQ ID NO: 29) |
| 6) | HDM2-M62A-2: | 5′-CATATAATCGTTTAGTCGCAATATACTGGCCAAG-3′ |
| | | (SEQ ID NO: 30) |
| 7) | petF2: | 5′-CATCGGTGATGTCGGCGAT-3′ |
| | | (SEQ ID NO: 3) |
| 8) | petR: | 5′-CGGATATAGTTCCTCCTTTCAGCA-3′ |
| | | (SEQ ID NO: 4) |
| 9) | h_p21_Forward: | 5′-GAGGCCGGGATGAGTTGGGAGGAG-3′ |
| | | (SEQ ID NO: 31) |
| 10) | h_p2l_Reverse: | 5′-CAGCCGGCGTTTGGAGTGGTAGAA-3′ |
| | | (SEQ ID NO: 32) |
| 11) | h_p53_forward: | 5′-CCCCTCCTGGCCCCTGTCATCTTC-3′ |
| | | (SEQ ID NO: 33) |
| 12) | h_p53_Reverse: | 5′-GCAGCGCCTCACAACCTCCGTCAT-3′ |
| | | (SEQ ID NO: 34) |
| 13) | h_b-actin_forward: | 5′-TCACCCACACTGTGCCCATCTACGA-3′ |
| | | (SEQ ID NO: 35) |
| 14) | h_b-actin_reverse: | 5′-CAGCGGAACCGCTCATTGCCAATGG-3′ |
| | | (SEQ ID NO: 36) |
| 15) | h_Gadd45alpha_forward: | 5′-GAGAGCAGAAGACCGAAAGGA-3′ |
| | | (SEQ ID NO: 37) |
| 16) | h_Gadd45alpha_reverse: | 5′-CAGTGATCGTGCGCTGACT-3′ |
| | | (SEQ ID NO: 38) |
| 17) | h_14-3-3sigma_forward: | 5′-ACTACGAGATCGCCAACAGC-3′ |
| | | (SEQ ID NO: 39) |
| 18) | h-14-3-3sigma_reverse: | 5′-CAGTGTCAGGTTGTCTCGCA-3′ |
| | | (SEQ ID NO: 40) |

### Vector Construction

Single mutant HDM2 clones were generated by Quickchange mutagenesis (Stratagene) of parental HDM2-PET22b using appropriate primers 1-6. The constructs were amplified with primers petF2 and petR to make HDM2 amplicons with T7 promoter and ribosome binding site required for in vitro transcription-translation (IVT) of wild-type or mutant HDM2. Primers 1-6 were used to introduce mutations into the parental pCMV-HDM2 mammalian expression construct by Quickchange mutagenesis. Both the HDM2-PET22b and pCMV-HDM2 constructs additionally encode a C-terminal HA tag. The plasmid p53-PET22b was also amplified with petF2 and petR to make template for IVT of wild-type p53.

### Immunoprecipitation and Western Blot Analysis

Protein G beads (Invitrogen) were incubated with anti-HA (1 μg per 10 μL beads) for 1 hour in PBST-3% BSA and subsequently washed twice in PBST-0.1% BSA. IVT expressed wild-type or mutant HDM2 was incubated with the beads on a rotator for 30 mins. Nutlin or stapled peptides were added at required concentrations and incubation carried out for 30 mins. IVT-expressed p53 was added to the mixture and incubation allowed for 1 hour. Beads were finally washed thrice in PBST-0.1% BSA and thrice with PBS, and bound proteins eluted by resuspension in 20 μL SDS-PAGE loading buffer and incubation at 95° C. for 5 minutes. Both the eluates and inputs were subjected to electrophoresis, transferred to nitrocellulose membranes and probed for p53 with Horse radish peroxidise conjugated DO1 antibody (Santa Cruz) or for HDM2 with anti-HA antibody followed by rabbit anti-mouse (Dakocytomation).

### Cell Culture

Mouse embryonic fibroblast p53/Mdm2 double-knockout (DKO) cells (a kind gift from Guillermina Lozano) [68] and H1299 p53^{-/-} cells [69] were maintained in Dulbecco's modified Eagle's medium (DMEM) with 10% (v/v) foetal calf serum (FCS) and 1% (v/v) penicillin/streptomycin. The cells were seeded at 1.0×10⁵ cells/well in 6-well plates, 24 hours prior to transfection. Cells were co-transfected with wild-type or mutant HDM2 plasmid, p53-pcDNA plasmid, LacZ reporter plasmid and luciferase plasmid using TurboFect transfection reagent (Thermo Scientific) according to the manufacturer's instructions. HCT116 p53^{+/+} cells [70] were maintained in McCoy's 5A medium with 10% (v/v) foetal calf serum (FCS) and 1% (v/v) penicillin/streptomycin. The cells were seeded at 3.5×10⁵ cells/well in 6-well plates, 24 hours prior to transfection. Cells were transfected with wild-type or mutant HDM2 plasmid using lipofectamine (Invitrogen) according to the manufacturer's instructions. Nutlin or stapled peptides were added to selected wells at required concentrations 4.5 hours post-transfection. In all cases, the total amount of plasmid DNA transfected per well was equilibrated by addition of the parental vector pcDNA3.1a(+).

### β-Galactosidase Assay and Western Blot Analysis

DKO cells were harvested 24 hours after transfection and β-galactosidase activities were assessed using the Dual-light System (Applied Biosystems) according to the manufacturer's protocol. The β-galactosidase activity was normalized with luciferase activity for each sample. To check for expression levels of relevant proteins via western blot, 5 μg of the cell lysates were probed for p53 with horseradish peroxidise conjugated DO1 antibody, for HDM2 and actin with anti-HA antibody and AC15 antibody respectively followed by rabbit anti-mouse.

### Protein Expression and Purification

DNA encoding HDM2 (amino acids 1-125) was ligated into the GST fusion expression vector pGEX-6P-1 (GE Lifesciences) via BamH1 and Nde1 double digest. Mutants of HDM2 (P20L, Q24R and M62A) were made using the QuickChange site-directed mutagenesis kit (Strategene) and appropriate primers 1-6. BL21 DE3 competent bacteria were then transformed with the GST tagged HDM2 (1-125) constructs. Cells harbouring the GST fusion constructs were grown in LB medium at 37° C. to an OD600 of ˜0.6 and induction was carried out with 1 mM IPTG at room temperature. Cells were harvested by centrifugation and the cell pellets were resuspended in 50 mM Tris pH 8.0, 10% sucrose and then sonicated. The sample was next centrifuged for 60 mins at 17,000 g at 4° C. The supernatant was applied to a 5 mL FF GST column (Amersham) pre-equilibrated in wash buffer (50 mM Tris-HCl pH 8.0, 150 mM NaCl, 1 mM DTT). The column was then further washed by 6 volumes of wash buffer. HDM2 constructs were then purified from the column by cleavage with Precission protease (GE Lifesciences). 10 units of Precission protease, in one column volume of wash buffer, were injected onto the column. The cleavage reaction was allowed to proceed overnight at 4° C. The cleaved protein was then eluted off the column with wash buffer. Protein fractions were analyzed with SDS page gel and concentrated using Centricon (3.5 kDa MWCO) concentrator. The protein samples were then dialyzed into buffer A solution (20 mM Bis-Tris, pH 6.5, 1 mM DTT) using HiPrep 26/10 Desalting column, and loaded onto a ResourceS 1 mL column pre-equilibrated in buffer A. The column was then washed in 6 column volumes of buffer A and bound protein was eluted with a linear gradient of 1 M NaCL over 30 column volumes. Protein fractions were analyzed with SDS page gel and concentrated using a Centricon (3.5 kDa MWCO) concentrator, Millipore. The cleaved HDM2 constructs were purified to ˜90% purity. Protein concentration was determined using A280 with extinction coefficients of 10430 M₋₁ cm₋₁ for the HDM2 (1-125) constructs.

mRNA Quantification

Total RNA was prepared from appropriately treated HCT116 p53^{+/+} cells using the RNeasy Mini Kit (QIAGEN). Reverse transcription was performed using SuperScript™ First-Strand Synthesis System (Invitrogen) with random hexamers. Realtime PCR assays (with appropriate primers 9-18) were carried out using the iQ SYBR Green Supermix (Bio-Rad) on the Bio-Rad CFX384 real-time PCR detection system. Experimental Ct values were normalized to β-actin and relative mRNA expression was calculated versus a reference sample. Data is shown as fold change in gene expression by RT-qPCR (Δ^{Δ}Ct method).

### Fluorescence Anisotropy

Apparent Kds of Nutlin and stapled peptides were determined by fluorescence anisotropy as previously described [24] using purified HDM2 (1-125) and carboxyfluorescein (FAM) labeled 12-1 peptide (FAM-RFMDYWEGL-NH2) [71]. Readings were carried out using the Envision Multilabel Reader (PerkinElmer). All experiments were carried out in PBS (2.7 mM KCl, 137 mM NaCl, 10 mM Na₂HPO₄ and 2 mM KH₂PO⁴ (pH 7.4)), 3% DMSO and 0.1% Tween 20 buffer. All titrations were carried out in triplicate. Curve-fitting was carried out using Prism 4.0 (GraphPad).

### F2H Co-Localization Assay

Transgenic BHK cells [27] were co-transfected with plasmids encoding the bait p53 (amino acids 1-81) fusion protein and different prey HDM2 (amino acids 7-134) fusion proteins overnight in 96 multiwell plates (uClear Greiner Bio-One, Germany) using the Lipofectamine 2000 (Life Technologies) reverse transfection protocol according to manufacturer's instructions with 0.2 μg DNA and 0.4 μL Lipofectamine 2000 per well. Cells were incubated with a dilution series of Nutlin or stapled peptides for 1 hour at 37° C., 5% CO₂.

Interaction (%) was determined as the ratio of cells showing co-localization of fluorescent signals at the nuclear spot to the total number of evaluated cells. For automated image acquisition an INCell Analyzer 1000 with a 20× objective (GE Healthcare) was used. Automated image segmentation and analysis was performed with the corresponding INCell Workstation 3.6 software. At least 100 co-transfected cells were analyzed per well. Titrations were carried out independently three to five times.

### In Silico Simulation Studies

The mutations P20L and Q24R lie in the flexible lid region of HDM2 and are missing from the crystal structures of HDM2 (1YCR, 1 RV1) [19,38]. In order to examine the dynamics of the full N-terminal region of HDM2, 11 conformations of the lid (residues 1-24) from the ensemble of NMR structures (1Z1M) [33] were grafted onto 1YCR (residues 25-109). The 11 structures were chosen visually to represent the 3 major states: open, closed and partially open. In addition, there is a recent crystal structure of HDM2 (residues 6-109) that has become available (in complex with a small molecule; PDB code 4HBM, resolved at 1.9 Å) [72] with an ordered lid and so a 12^{th} structure of 1YCR was also created where this lid (only from residues 6-24) was grafted. The 12 structures generated (wild-type) and the P20L and Q24R mutants generated were all subject to 20 ns molecular dynamics simulations each, in 3 states: apo, complex with p53 peptide and complex with Nutlin, totaling a simulation time of 720 ns for the wild type and for each mutant. A shorter HDM2 was used for HDM2-stapled peptide complexes. For this, residues 19-24 of HDM2 were crafted from 4ERF resolved at 2.0 Å [46] on to 1YCR, so as the initial structure include residues 19-109 of HDM2. The staple was built using Xleap module in Amber and the parameters were derived from antechamber [57,73] module in Amber. Nutlin parameters were also derived using antechamber. Molecular dynamics simulations were performed with the SANDER module of the AMBER11 [55] package employing the all-atom Cornell force field [56]. Simulations were also carried out for p53, PM2 and M011 peptides bound to HDM2 (19-109) and its mutants (Q24R and M62A). All systems were prepared as described before [16] and simulated for 100 ns at constant temperature (300K) and pressure (1 atm) and structures were stored every 10 ps. The computational alanine-scanning methodology [74] is based on the assumption that replacing the original residue with an alanine will only introduce local changes and not cause a large conformational change to alter the binding mode. Trajectories were sampled every 100 ps for computational alanine scanning using the MM-PBSA post-processing module in amber11. Alanine mutant structures were generated by modifying each residue of the receptor at the C_{γ} atom and by replacing the C_{γ} atom with a hydrogen atom with appropriate distance at the C_{γ}-C_{β} bond. PyMOL [75] and Visual Molecular Dynamics [76] (VMD) were used for visualizations.

### Statistical Analysis

For the F2H assay, significance (t-test) is denoted relative to the percentage interactions observed for wild-type HDM2 under the indicated treatment conditions. For transcript analysis by real-time PCR, two-way ANOVA with Bonferroni post test was performed using GraphPad Prism software.

### Results

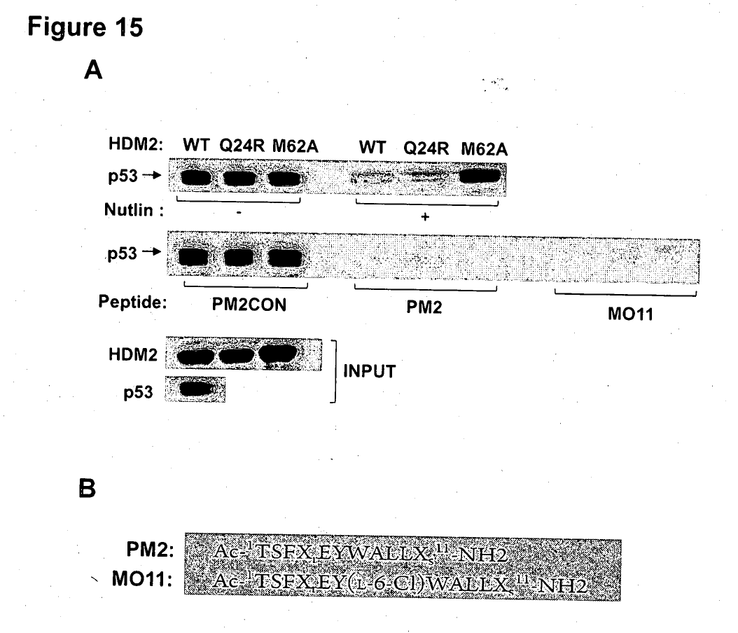

Pull-down assays were first carried out using in vitro expressed proteins to investigate disruption of the HDM2-p53 interaction by Nutlin and the stapled peptides PM2 and MO11 (FIG. 15) [24]. These have been designed to target the same hydrophobic cleft of HDM2 to which Nutlin binds. Either wild-type or mutant (M62A and Q24R) HDM2 was captured on beads followed by incubation with either Nutlin or stapled peptide. p53 was subsequently added, and interaction with HDM2 determined by Western blot. The results in FIG. 15 indicate strong repression of the HDM2-p53 interaction by both Nutlin and the stapled peptides. As previously described, the M62A and Q24R mutants showed resistance to Nutlin, with increased p53 being pulled down compared to wildtype HDM2 [77]. In striking comparison, the stapled peptides PM2 and MO11 were able to abrogate the mutant HDM2-p53 interaction as efficiently as Nutlin inhibits the wildtype HDM2-p53 interaction. A control stapled peptide PM2CON (PM2 with 3 critical contact residues mutated to alanine) had no effect on binding of p53 to HDM2.

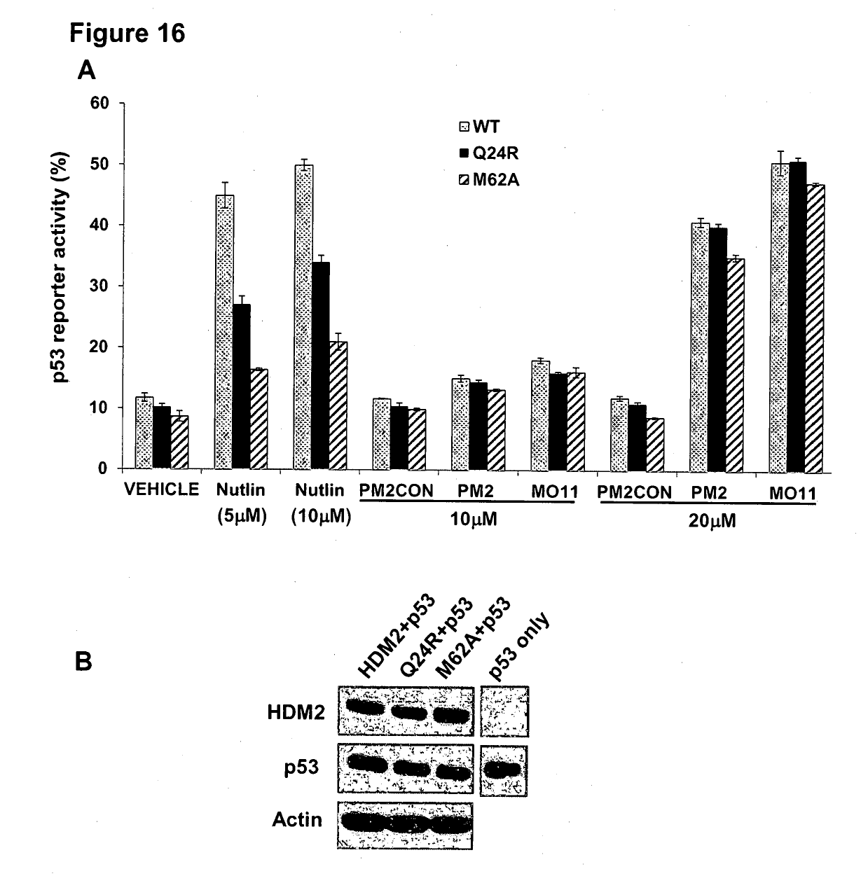

Reporter assays in the p53/MDM2-null DKO cell line were carried out to measure p53 transactivation function in the presence of HDM2 and Nutlin/stapled peptides. The results show a clear difference in the ability of Nutlin and the stapled peptides to antagonize mutant HDM2 function (FIG. 16). In the absence of antagonist, p53 function was reduced ˜90% by wild-type and mutant HDM2. Addition of Nutlin (10 μM) restored p53 activity to 50% that seen in absence of HMD2 co-transfection. For the Nutlin resistant Q24R and M62A mutants, activity was restored to only 34% and 21% respectively. In contrast, the stapled peptides behaved essentially like Nutlin in disruption of the wild-type HDM2-p53 interaction at the higher dose tested (20 μM). PM2 restored activity to 41% whilst the more potent MO11 restored activity to 51%. Notably, the stapled peptides were able to efficiently antagonize the HDM2 mutants. In the case of Q24R, activity was restored to the same level as for inhibition of wild-type HDM2. For M62A, activity was restored to 35% and 47% by PM2 and MO11, respectively.

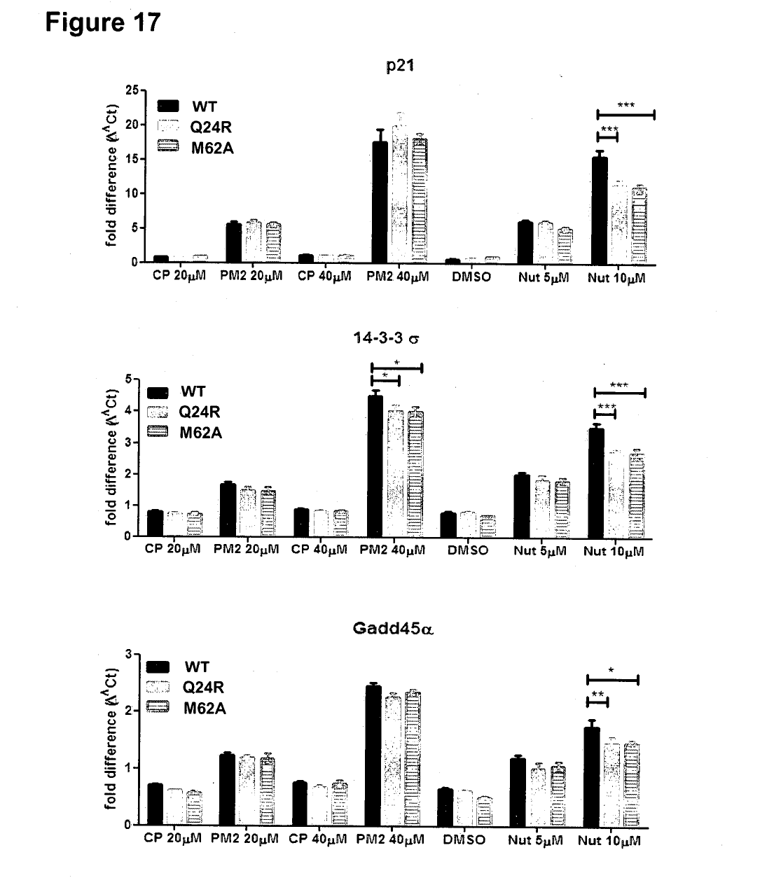

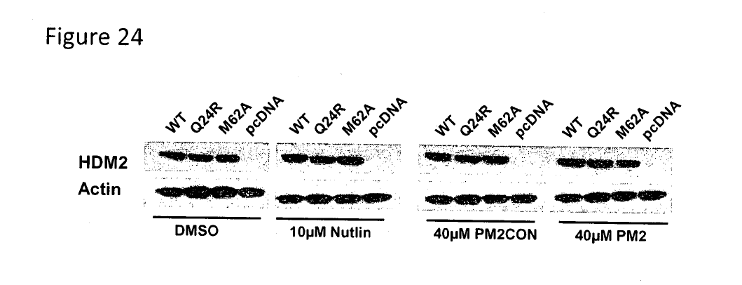

The behavior of the different ligands with respect to regulation of endogenous p53-dependent genes was next investigated in HCT116 p53^{+/+} cells (FIG. 17). Wild-type or variant HDM2 was transfected and cells treated with either Nutlin or stapled peptide PM2. p53 activation of p21, gadd45α and 14-3-3σ transcript levels [78] [79,80] was measured by qPCR. In the case of Nutlin treatment (10 μM), significant reduction of p53 transcriptional activity was observed for the M62A and Q24R mutants compared to wild-type, consistent with results obtained in DKO cells. The stapled peptide PM2 (40 μM) did not discriminate significantly between inhibition of wild-type and mutant HDM2 with regards to up-regulation of the p21 and Gadd45α genes. In the case of the 14-3-3σ gene, some resistance to PM2 was observed for the mutants, although this was not as pronounced when compared to Nutlin treatment. No significant differences in expression of the HDM2 mutants were observed compared to wild-type in this cell line (FIG. 24).

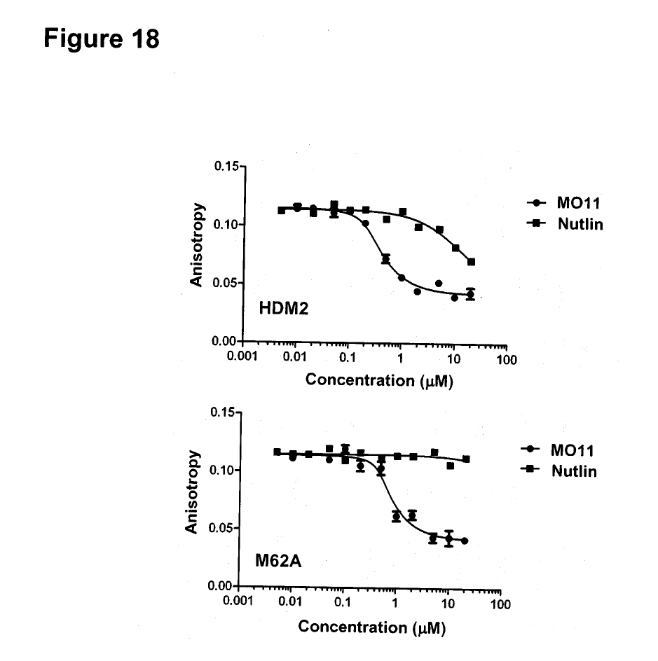

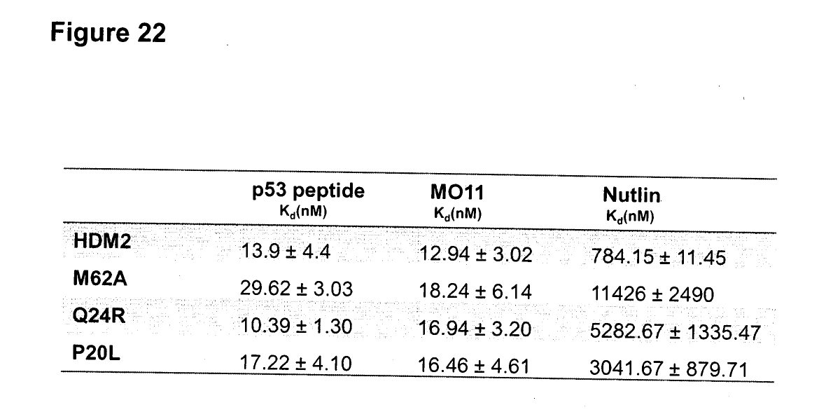

Affinity measurements indicated that the M62A mutation significantly reduced affinity for Nutlin compared to wild-type (11426±2490 versus 784.15±11.45 nM respectively)(FIGS. 18, 22). In contrast, very slight perturbation of binding to p53 peptide (29.62±3.03 versus 13.9±4.4 nM for wild-type) and stapled peptide MO11 (18.24±6.14 versus 12.94±3.02 nM for wild-type) was observed. The Q24R and P20L mutants also displayed reduced affinity for Nutlin compared to wild-type (respectively 5282.67±1335.47 and 3041.67±879.71 versus 784.15±11.45 nM). The trend in Nutlin binding affinity for the mutants (M62A<Q24R<P20L) mirrors the resistance phenotypes observed for these mutants in cell-based assays (FIGS. 16, 17) [77]. Binding to the stapled peptide MO11 was not perturbed by the Q24R and P20L mutations (respectively 16.94±3.20 and 16.46±4.61 versus 12.94±3.02 nM for wild-type). Similarly, no significant differences were observed for p53 peptide binding to these mutants (10.39±1.30 and 17.22±4.10 versus 13.9±4.4 nM for wild-type).

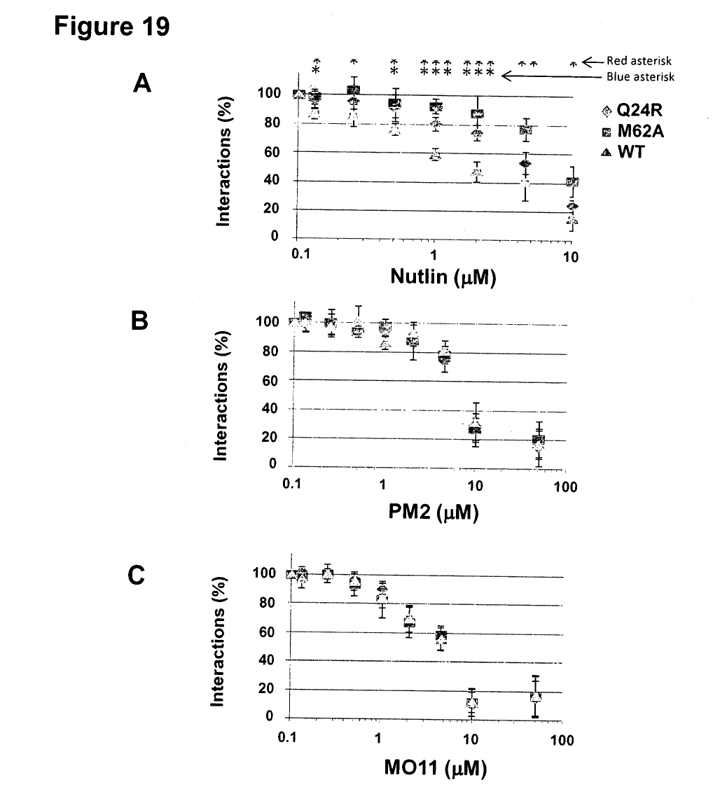

The direct cellular binding of the HDM2 wild-type, Q24R and M62A N-terminal domains to p53 was further characterized in the Fluorescent 2-Hybrid (F2H) assay [27]. The F2H assay visualizes the interaction of RFP-tagged HDM2 (amino acids 7-134) with GFP-tagged p53 (amino acids 1-81) at a defined nuclear F2H interaction platform, in specific BHK cells. Dissociation of the complex due to interaction with Nutlin or stapled peptide can be imaged and quantified. Compared to the wild-type HDM2-p53 interaction, addition of Nutlin resulted in reduced dissociation of mutant N-terminal domains from p53, indicating Nutlin resistance (FIG. 19). This was particularly evident in the dose range 1-10 μM. In comparison, no significant differences were seen between wild-type and mutant N-terminal domains when the stapled peptides were used to dissociate the complexes. In agreement with the reporter assays (FIG. 16), MO11 was more potent than PM2 in disrupting the complex.

### Discussion

Systematic analysis of small molecule versus peptide binding to target proteins indicates that the former do not take advantage of all the available opportunities for polar contacts, and typically rely on a few anchor points and hydrophobic interactions to achieve high-potency binding [81]. This binding deficit may therefore be readily exploited through point mutation, as seen with the M62A and Q24R mutations in HDM2. In contrast, the peptide-protein binding interface generally employs a more diffuse network of polar interactions, intimating that peptide/peptide-like molecules should be intrinsically more recalcitrant to point mutations in target proteins. The present data supports this notion, as both in vitro and ex vivo assays indicate that point mutants of HMD2 that inhibit Nutlin, but not p53 binding have reduced or no impact on the interaction with stapled peptides. As these mutants were originally selected to retain p53, but not Nutlin binding [77], this shows that the stapled peptides faithfully mimic the endogenous p53 N-terminal domain interaction with HDM2. Further selections are currently under way to determine whether HDM2 resistance to the stapled peptides (but not p53) can be evolved. Based on in silico predictions (see below), it is likely that a higher mutational burden will be required. Furthermore, in the context of the overall p53-HDM2 binding interaction, it is plausible that mutations in the secondary binding interface that selectively increase affinity for p53 (for example V280A [77]) could indirectly confer resistance to stapled peptides. Given the highly allosteric nature of HDM2 [40], mutations in distal domains may also impact on binding of stapled peptides.

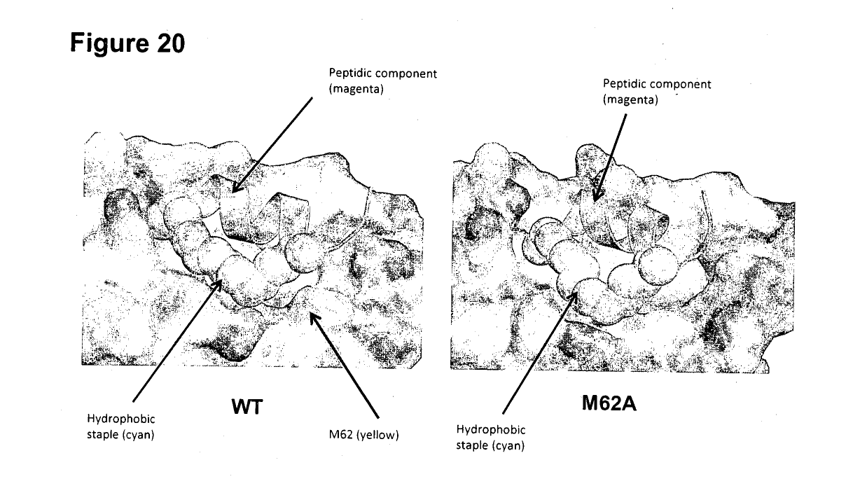

Modelling studies show the hydrophobic hydrocarbon chain comprising the staple interacts with HDM2 in the vicinity of M62 (FIG. 20). Whilst the M62A mutation impacts negatively on binding of both stapled peptide and p53 compared to wild type HDM2, the presence of additional “fall-back” interactions (apart from M62, see below) results in marginal overall loss of binding by these ligands. In the case of Nutlin binding, interaction with M62 contributes significantly to overall binding, and hence major loss of binding occurs when this contact is lost [77].

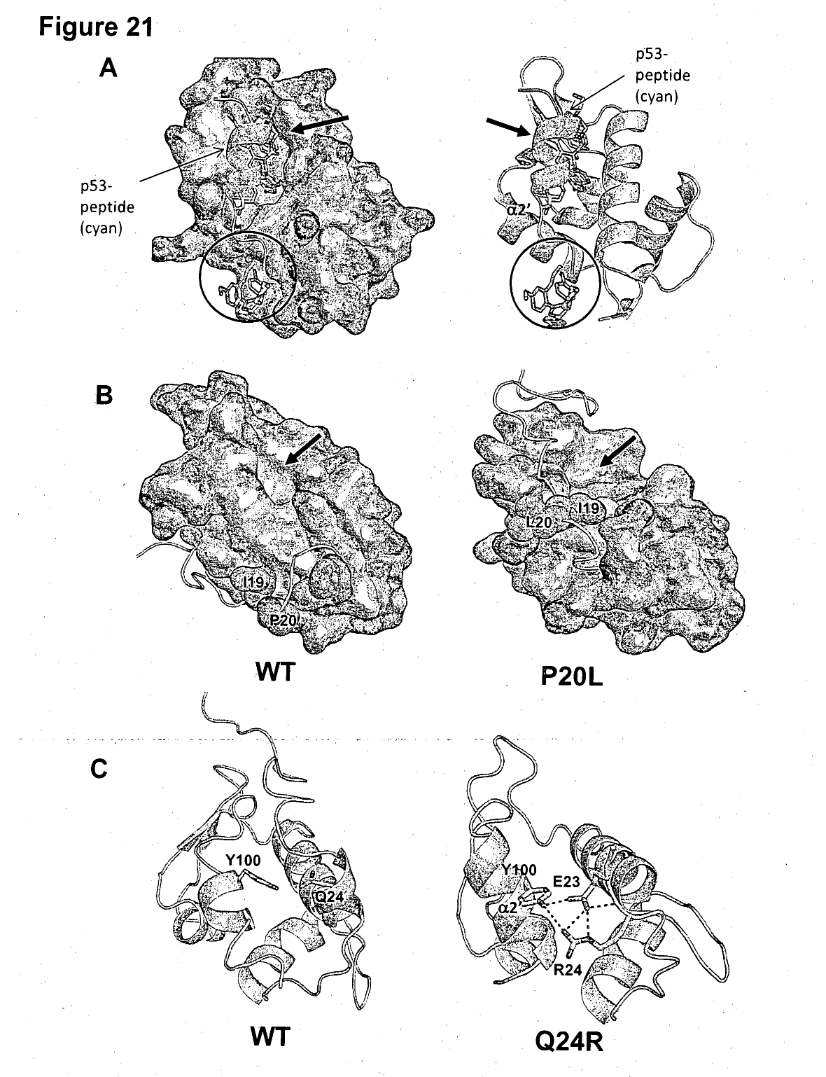

To date, most understanding of the interactions of Nutlin with HDM2 have focused on the “main” site (FIG. 21A), and indeed this has been instrumental in the design of small molecules that are now in clinical trials. However, the crystal structure of Nutlin complexed with HDM2 also shows a second molecule of Nutlin that interacts with the α2′ region of HDM2, which is called the “secondary” site (FIG. 21A). This interaction has never been deemed important as it was thought to result from crystal contacts. Recently, hints that there may be more to this appeared when Brownian Dynamics simulations demonstrated that in solution, Nutlin would bind to this site also [82]. More recently, H-D exchange data combined with molecular simulations and rationally designed mutagenesis studies have demonstrated that this region near α2′, is the site where Nutlin appears to first bind and then shuttle to the “main” binding site [83]. Simulations of the P20L mutation shows that L20 together with 119 packs against the ridge of the p53 binding pocket leading to a partial occlusion of the main binding site (FIG. 21B), notably the region where L26 of p53 embeds. Cluster analysis on the apo P20L MD data shows that 84% of the conformations sampled place the 119-L20 in this position within HDM2. This places a barrier for Nutlin migration from the secondary to the main site and may account for the resistance of this mutant to Nutlin binding.

Binding to p53 is retained as the lid only occludes the L26 site; it has previously been shown that p53 likely binds with F19 docking first and enabling a crack to propagate [84]. This suggests that in these mutants, p53 and stapled peptides can dock into the open F19 docking site and then slowly edge the lid out.

The Y104G mutation in the secondary Nutlin binding site is recalcitrant to Nutlin binding (yet retains p53 binding), thus suggesting that this binding site indeed may be crucial for Nutlin interactions as hypothesized [83]. Earlier Brownian dynamics simulations have also provided hints that residues in this region (E25 and K51) appear to play key roles in channeling Nutlin into HDM2 [84]. These residues (E25, K51 and Y104) are in closer proximity to the second Nutlin binding site than the primary p53/Nutlin binding site. Studies have shown that K51 of HDM2 interacts with E23 and E25 of HDM2 [85]. Simulations indicate that the Q24R mutation leads to the development of a cationic potential in the region of R24 and will therefore undoubtedly influence the dynamics of E25. In the simulations of Q24R, it is clear that it engages in an extensive hydrogen bond network with E23 and Y100, all in the vicinity of the secondary site (FIG. 21C). This has two effects that will likely deter Nutlin “landing”: destabilizing the E25-K51 salt bridge which perturbs the secondary site and interactions of R24 with E23, Y100 which occludes the secondary site; the conformations that are sampled account for ˜42% of the total sample.

Many computational studies have been used to address resistance mutations and their structural and energetic coupling to inhibitors in EGFR kinase [86] and HIV [87,88]. However, it would be useful if a method could predict the emergence of mutations at key sites in proteins.

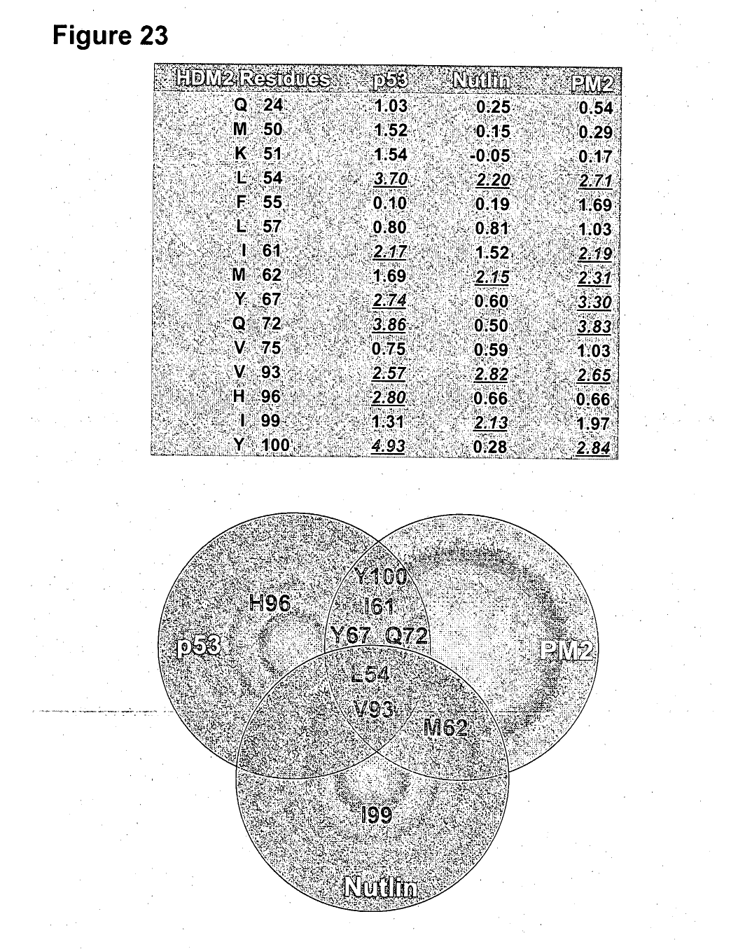

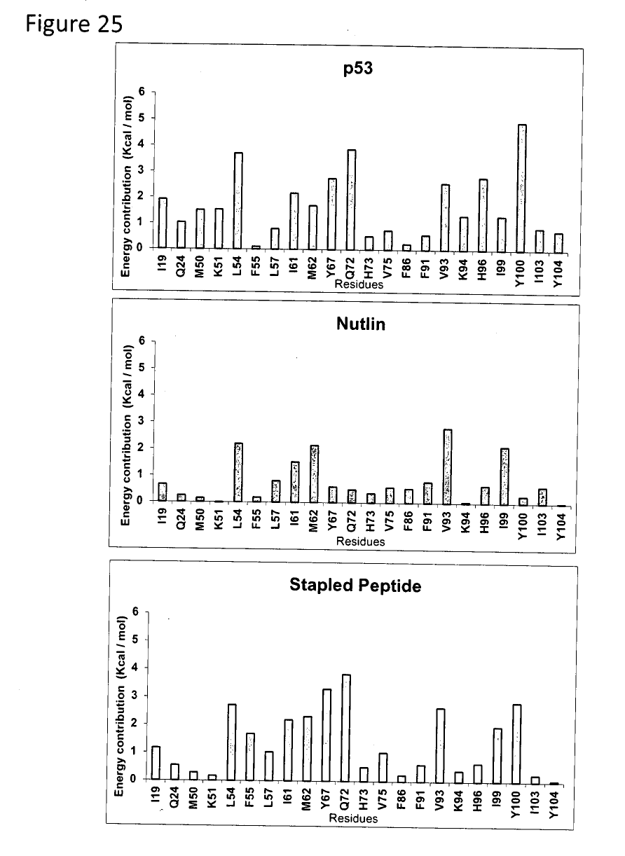

Towards this end, it was investigated what mutation would enable HDM2 to destabilize interactions with stapled peptide and strengthen them with p53. A computational alanine scan was therefore carried out whereby all the residues in the HDM2 N-terminal domain (except glycine, alanine and proline) were individually mutated to alanine. The effects of point mutations on the interactions with p53 peptide, Nutlin, and stapled peptide were recalculated for the absolute binding free energy for the mutated system. The computational alanine scanning results for selected residues are summarised in FIG. 23 and FIG. 25. Positive and negative values indicate unfavourable and favourable contributions, respectively. Seven residues contribute significantly (>2 kcal/mol) to binding of p53 peptide and PM2, of which 6 are common to both ligands (FIG. 23). The multiplicity of shared anchor points indicates that point mutations selectively discriminating against stapled peptide, but not p53 binding are less probable. In contrast, only four residues (L54, M62, V93, 199) contribute significantly to Nutlin binding. Of these, L54 and V93 are important for binding of all ligands, whilst M62 plays a significant role for PM2 and Nutlin binding. Hence, as shown experimentally for M62, mutation of any of the residues important for Nutlin binding is likely to selectively perturb Nutlin but not p53 binding to HDM2. Whilst M62 is also involved in binding of stapled peptide (FIG. 20), the presence of numerous other contact points results in no significant detriment to binding when this amino acid is mutated. It is important to note that this analysis does not account for whether the mutations destabilise the HDM2 fold. However, such mutations would most likely impact negatively on p53 binding, and are thus unlikely to arise. Computational predictions therefore suggest that selective resistance to stapled peptides is unlikely to occur through point mutation in the N-terminal p53-binding pocket of HDM2. However, it is important to further query this hypothesis using both rational and directed evolution approaches.

In this study, it was demonstrated that stapled p53-peptide analogues can function in cells as next-generation ligands capable of reverting a drug-resistant phenotype due to mutation in HDM2. As small molecule HDM2 inhibitors have yet to be approved for clinical use, it remains to be seen whether the resistance mutations identified will manifest. Furthermore, one cannot discount acquired resistance to stapled-peptide analogues should these prove viable therapeutic reagents. In this case, application of both guided and combinatorial selection methods will expedite the development of second-line antagonists.

### Applications

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### REFERENCES

1. Kastan M B, Onyekwere O, Sidransky D, Vogelstein B, Craig R W (1991) Participation of p53 protein in the cellular response to DNA damage. Cancer Res 51: 6304-6311.
2. Kuerbitz S J, Plunkett B S, Walsh W V, Kastan M B (1992) Wild-type p53 is a cell cycle checkpoint determinant following irradiation. Proc Natl Acad Sci USA 89: 7491-7495.
3. Vogelstein B, Lane D, Levine A J (2000) Surfing the p53 network. Nature 408: 307-310.
4. Beroud C, Soussi T (2003) The UMD-p53 database: new mutations and analysis tools. Hum Mutat 21: 176-181.
5. Hollstein M, Sidransky D, Vogelstein B, Harris C C (1991) p53 mutations in human cancers. Science 253: 49-53.
6. Kan Z, Jaiswal B S, Stinson J, Janakiraman V, Bhatt D, et al. (2010) Diverse somatic mutation patterns and pathway alterations in human cancers. Nature 466: 869-873.
7. Momand J, Zambetti G P, Olson D C, George D, Levine A J (1992) The mdm-2 oncogene product forms a complex with the p53 protein and inhibits p53-mediated transactivation. Cell 69: 1237-1245.
8. Haupt Y, Maya R, Kazaz A, Oren M (1997) Mdm2 promotes the rapid degradation of p53. Nature 387: 296-299.
9. Kubbutat M H, Jones S N, Vousden K H (1997) Regulation of p53 stability by Mdm2. Nature 387: 299-303.
10. Honda R, Tanaka H, Yasuda H (1997) Oncoprotein MDM2 is a ubiquitin ligase E3 for tumor suppressor p53. FEES Lett 420: 25-27.
11. Tamborini E, Della Torre G, Lavarino C, Azzarelli A, Carpinelli P, et al. (2001) Analysis of the molecular species generated by MDM2 gene amplification in liposarcomas. Int J Cancer 92: 790-796.
12. Schlott T, Reimer S, Jahns A, Ohlenbusch A, Ruschenburg I, et al. (1997) Point mutations and nucleotide insertions in the MDM2 zinc finger structure of human tumours. J Pathol 182: 54-61.
13. Bond G L, Hu W, Bond E E, Robins H, Lutzker S G, et al. (2004) A single nucleotide polymorphism in the MDM2 promoter attenuates the p53 tumor suppressor pathway and accelerates tumor formation in humans. Cell 119: 591-602.
14. Oliner J D, Kinzler K W, Meltzer P S, George D L, Vogelstein B (1992) Amplification of a gene encoding a p53-associated protein in human sarcomas. Nature 358: 80-83.
15. Zhou M, Gu L, Abshire T C, Homans A, Billett A L, et al. (2000) Incidence and prognostic significance of MDM2 oncoprotein overexpression in relapsed childhood acute lymphoblastic leukemia. Leukemia 14: 61-67.
16. Joseph T L, Madhumalar A, Brown C J, Lane D P, Verma C S (2010) Differential binding of p53 and nutlin to MDM2 and MDMX: computational studies. Cell Cycle 9: 1167-1181.
17. Fen C X, Coomber D W, Lane D P, Ghadessy F J (2007) Directed evolution of p53 variants with altered DNA-binding specificities by in vitro compartmentalization. J Mol Biol 371: 1238-1248.
18. Noureddine M A, Menendez D, Campbell M R, Bandele O J, Horvath M M, et al. (2009) Probing the functional impact of sequence variation on p53-DNA interactions using a novel microsphere assay for protein-DNA binding with human cell extracts. PLoS Genet 5: el000462.
19. Kussie P H, Gorina S, Marechal V, Elenbaas B, Moreau J, et al. (1996) Structure of the MDM2 oncoprotein bound to the p53 tumor suppressor transactivation domain. Science 274: 948-953.
20. Chen J, Marechal V, Levine A J (1993) Mapping of the p53 and mdm-2 interaction domains. Mol Cell Biol 13: 4107-4114.
21. Marechal V, Elenbaas B, Piette J, Nicolas J C, Levine A J (1994) The ribosomal L5 protein is associated with mdm-2 and mdm-2-p53 complexes. Mol Cell Biol 14: 7414-7420.
22. Elenbaas B, Dobbelstein M, Roth J, Shenk T, Levine A J (1996) The MDM2 oncoprotein binds specifically to RNA through its RING finger domain. Mol Med 2: 439-451.
23. Yu G W, Allen M D, Andreeva A, Fersht A R, Bycroft M (2006) Solution structure of the C4 zinc finger domain of HDM2. Protein Sci 15: 384-389.
24. Fang S, Jensen J P, Ludwig R L, Vousden K H, Weissman A M (2000) Mdm2 is a RING finger-dependent ubiquitin protein ligase for itself and p53. J Biol Chem 275: 8945-8951.
25. Parant J, Chavez-Reyes A, Little N A, Yan W, Reinke V, et al. (2001) Rescue of embryonic lethality in Mdm4-null mice by loss of Trp53 suggests a nonoverlapping pathway with MDM2 to regulate p53. Nat Genet 29: 92-95.
26. Funston G, Goh W, Wei S J, Tng Q S, Brown C, et al. (2012) Binding of Translationally Controlled Tumour Protein to the N-Terminal Domain of HDM2 Is Inhibited by Nutlin-3. PLoS One 7: e42642.
27. Zolghadr K, Mortusewicz O, Rothbauer U, Kleinhans R, Goehler H, et al. (2008) A fluorescent two-hybrid assay for direct visualization of protein interactions in living cells. Mol Cell Proteomics 7: 2279-2287.
28. Hu B, Gilkes D M, Farooqi B, Sebti S M, Chen J (2006) MDMX overexpression prevents p53 activation by the MDM2 inhibitor Nutlin. J Biol Chem 281: 33030-33035.
29. Gorre M E, Mohammed M, Ellwood K, Hsu N, Paquette R, et al. (2001) Clinical resistance to STI-571 cancer therapy caused by BCR-ABL gene mutation or amplification. Science 293: 876-880.
30. Yun C H, Mengwasser K E, Toms A V, Woo M S, Greulich H, et al. (2008) The T790M mutation in EGFR kinase causes drug resistance by increasing the affinity for ATP. Proc Natl Acad Sci USA 105: 2070-2075.
31. Gorre M E, Ellwood-Yen K, Chiosis G, Rosen N, Sawyers C L (2002) BCR-ABL point mutants isolated from patients with imatinib mesylate-resistant chronic myeloid leukemia remain sensitive to inhibitors of the BCR-ABL chaperone heat shock protein 90. Blood 100: 3041-3044.
32. McCoy M A, Gesell J J, Senior M M, Wyss D F (2003) Flexible lid to the p53-binding domain of human Mdm2: implications for p53 regulation. Proc Natl Acad Sci USA 100: 1645-1648.
33. Uhrinova S, Uhrin D, Powers H, Watt K, Zheleva D, et al. (2005) Structure of free MDM2 N-terminal domain reveals conformational adjustments that accompany p53-binding. J Mol Biol 350: 587-598.
34. Showalter S A, Bruschweiler-Li L, Johnson E, Zhang F, Bruschweiler R (2008) Quantitative lid dynamics of MDM2 reveals differential ligand binding modes of the p53-binding cleft. J Am Chem Soc 130: 6472-6478.
35. Worrall E G, Worrall L, Blackburn E, Walkinshaw M, Hupp T R (2010) The effects of phosphomimetic lid mutation on the thermostability of the N-terminal domain of MDM2. J Mol Biol 398: 414-428.
36. Worrall E G, Wawrzynow B, Worrall L, Walkinshaw M, Ball K L, et al. (2009) Regulation of the E3 ubiquitin ligase activity of MDM2 by an N-terminal pseudo-substrate motif. J Chem Biol 2: 113-129.
37. Yu G W, Rudiger S, Veprintsev D, Freund S, Fernandez-Fernandez M R, et al. (2006) The central region of HDM2 provides a second binding site for p53. Proc Natl Acad Sci USA 103: 1227-1232.
38. Vassilev L T, Vu B T, Graves B, Carvajal D, Podlaski F, et al. (2004) In vivo activation of the p53 pathway by small-molecule antagonists of MDM2. Science 303: 844-848.
39. Ma J, Martin J D, Zhang H, Auger K R, Ho T F, et al. (2006) A second p53 binding site in the central domain of Mdm2 is essential for p53 ubiquitination. Biochemistry 45: 9238-9245.
40. Wawrzynow B, Pettersson S, Zylicz A, Bramham J, Worrall E, et al. (2009) A function for the RING finger domain in the allosteric control of MDM2 conformation and activity. J Biol Chem 284: 11517-11530.
41. Bothner B, Lewis W S, DiGiammarino E L, Weber J D, Bothner S J, et al. (2001) Defining the molecular basis of Arf and Hdm2 interactions. J Mol Biol 314: 263-277.
42. Sivakolundu S G, Nourse A, Moshiach S, Bothner B, Ashley C, et al. (2008) Intrinsically unstructured domains of Arf and Hdm2 form bimolecular oligomeric structures in vitro and in vivo. J Mol Biol 384: 240-254.
43. Lohrum M A, Ludwig R L, Kubbutat M H, Hanlon M, Vousden K H (2003) Regulation of HDM2 activity by the ribosomal protein L11. Cancer Cell 3: 577-587.
44. Zhang Y, Wolf G W, Bhat K, Jin A, Allio T, et al. (2003) Ribosomal protein L11 negatively regulates oncoprotein MDM2 and mediates a p53-dependent ribosomal-stress checkpoint pathway. Mol Cell Biol 23: 8902-8912.
45. Lindstrom M S, Jin A, Deisenroth C, White Wolf G, Zhang Y (2007) Cancer-associated mutations in the MDM2 zinc finger domain disrupt ribosomal protein interaction and attenuate MDM2-induced p53 degradation. Mol Cell Biol 27: 1056-1068.
46. Rew Y, Sun D, Gonzalez-Lopez De Turiso F, Bartberger M D, Beck H P, et. al. (2012) Structure-based design of novel inhibitors of the MDM2-p53 interaction. J Med Chem 55: 4936-4954.
47. Bernal F, Tyler A F, Korsmeyer S J, Walensky L D, Verdine G L (2007) Reactivation of the p53 tumor suppressor pathway by a stapled p53 peptide. J Am Chem Soc 129: 2456-2457.
48. Baek S, Kutchukian P S, Verdine G L, Huber R, Holak T A, et al. (2012) Structure of the stapled p53 peptide bound to Mdm2. J Am Chem Soc 134: 103-106.
49. Garraway L A, Janne P A (2012) Circumventing cancer drug resistance in the era of personalized medicine. Cancer Discov 2: 214-226.
50. Guo T, Hajdu M, Agaram N P, Shinoda H, Veach D, et al. (2009) Mechanisms of sunitinib resistance in gastrointestinal stromal tumors harboring KITAY502-3ins mutation: an in vitro mutagenesis screen for drug resistance. Clin Cancer Res 15: 6862-6870.
51. Azam M, Latek R R, Daley G Q (2003) Mechanisms of autoinhibition and STI-571/imatinib resistance revealed by mutagenesis of BCR-ABL. Cell 112: 831-843.
52. Emery C M, Vijayendran K G, Zipser M C, Sawyer A M, Niu L, et al. (2009) MEK1 mutations confer resistance to MEK and B-RAF inhibition. Proc Natl Acad Sci USA 106: 20411-20416.
53. Tawfik D S, Griffiths A D (1998) Man-made cell-like compartments for molecular evolution. Nat Biotechnol 16: 652-656.
54. Zaccolo M, Gherardi E (1999) The effect of high-frequency random mutagenesis on in vitro protein evolution: a study on TEM-1 beta-lactamase. J Mol Biol 285: 775-783.
55. D. A. Case, T. A. Darden, T. E. Cheatham I, C. L. Simmerling, J. Wang, et al. (2010) AMBER 11. University of California, San Francisco.
56. Cornell W D, Cieplak P, Bayly C I, Gould I R, Merz K M, et al. (1995) A second-generation force field for the simulation of proteins, nucleic acids, and organic molecules. J Am Chem Soc 117: 5179-5197.
57. Wang J, Wolf R M, Caldwell J W, Kollman P A, Case D A (2004) Development and testing of a general AMBER force field. J Comput Chem 25: 1157-1174.
58. de Vries S J, van Dijk M, Bonvin A M (2010) The HADDOCK web server for data-driven biomolecular docking. Nat Protoc 5: 883-897.
59. Raveh B, London N, Zimmerman L, Schueler-Furman 0 (2011) Rosetta FlexPepDock ab-initio: simultaneous folding, docking and refinement of peptides onto their receptors. PLoS One 6: e18934.
60. Walensky L D, Kung A L, Escher I, Malia T J, Barbuto S, et al. (2004) Activation of apoptosis in vivo by a hydrocarbon-stapled BH3 helix. Science 305: 1466-1470.
61. Kim Y W, Grossmann T N, Verdine G L (2011) Synthesis of all-hydrocarbon stapled alpha-helical peptides by ring-closing olefin metathesis. Nat Protoc 6: 761-771.
62. Verdine G L, Walensky L D (2007) The challenge of drugging undruggable targets in cancer: lessons learned from targeting BCL-2 family members. Clin Cancer Res 13: 7264-7270.
63. Phillips C, Roberts L R, Schade M, Bazin R, Bent A, et al. (2011) Design and structure of stapled peptides binding to estrogen receptors. J Am Chem Soc 133: 9696-9699.
64. Brown C J, Quah S T, Jong J, Goh A M, Chiam P C, et al. (2012) Stapled Peptides with Improved Potency and Specificity That Activate p53. ACS Chem Biol.
65. Lai Z, Auger K R, Manubay C M, Copeland R A (2000) Thermodynamics of p53 binding to hdm2(1-126): effects of phosphorylation and p53 peptide length. Arch Biochem Biophys 381: 278-284.
66. Schon O, Friedler A, Bycroft M, Freund S M, Fersht A R (2002) Molecular mechanism of the interaction between MDM2 and p53. J Mol Biol 323: 491-501.
67. Dastidar S G, Lane D P, Verma C S (2008) Multiple peptide conformations give rise to similar binding affinities: molecular simulations of p53-MDM2. J Am Chem Soc 130: 13514-13515.
68. Montes de Oca Luna R, Wagner D S, Lozano G (1995) Rescue of early embryonic lethality in mdm2-deficient mice by deletion of p53. Nature 378: 203-206.
69. Takahashi T, Carbone D, Nau M M, Hida T, Linnoila I, et al. (1992) Wild-type but not mutant p53 suppresses the growth of human lung cancer cells bearing multiple genetic lesions. Cancer Res 52: 2340-2343.
70. Boyer J, McLean E G, Aroori S, Wilson P, McCulla A, et al. (2004) Characterization of p53 wild-type and null isogenic colorectal cancer cell lines resistant to 5-fluorouracil, oxaliplatin, and irinotecan. Clin Cancer Res 10: 2158-2167.
71. Bottger V, Bottger A, Howard S F, Picksley S M, Chene P, et al. (1996) Identification of novel mdm2 binding peptides by phage display. Oncogene 13: 2141-2147.
72. Michelsen K, Jordan J B, Lewis J, Long A M, Yang E, Rew Y, Zhou J, Yakowec P, Schnier P D, Huang X, Poppe L. (2012) Ordering of the N-terminus of human MDM2 by small molecule inhibitors. J Am Chem Soc 134: 17059-17067.
73. Wang J, Wang, W., Kollman P A, Case D A, (2006) Automatic atom type and bond type perception in molecular mechanical calculations. J Mol Graph Model 25: 247-260.
74. Massova I, Kollman, P A., (1999) Computational alanine scanning to probe protein-protein interactions: A novel approach to evaluate binding free energies. J Am Chem Soc 121: 8133-8143.
75. DeLano W L, (2002) The PyMOL molecular graphics system. San Carlos, Calif., USA: DeLano Scientific.
76. Humphrey W, Dalke A, Schulten K. (1996) VMD-visual molecular dynamics. J Mol Graph Model 14: 33-38.
77. Wei S J, Joseph T, Sim A Y, Yurlova L, Zolghadr K, et al. (2013) In Vitro Selection of Mutant HDM2 Resistant to Nutlin Inhibition. PLoS One 8: e62564.
78. Kastan M B, Zhan Q, el-Deiry W S, Carrier F, Jacks T, et al. (1992) A mammalian cell cycle checkpoint pathway utilizing p53 and GADD45 is defective in ataxia telangiectasia. Cell 71: 587-597.
79. el-Deiry W S, Tokino T, Velculescu V E, Levy D B, Parsons R, et al. (1993) WAF1, a potential mediator of p53 tumor suppression. Cell 75: 817-825.
80. Hermeking H, Lengauer C, Polyak K, He T C, Zhang L, et al. (1997) 14-3-3 sigma is a p53-regulated inhibitor of G2/M progression. Mol Cell 1: 3-11.
81. Higueruelo A P, Schreyer A, Bickerton G R, Blundell T L, Pitt W R (2012) What can we learn from the evolution of protein-ligand interactions to aid the design of new therapeutics? PLoS One 7: e51742.
82. ElSawy K M, Verma C S, Joseph T L, Lane D P, Twarock R, Caves L S. (2013) On the interaction mechanisms of a p53 peptide and nutlin with the MDM2 and MDMX proteins: a Brownian dynamics study. Cell Cycle 12: 394-404.
83. Hernychova L, Man P, Verma C, Nicholson J, Sharma C A, Ruckova E, Teo J Y, Ball K, Vojtesek B, Hupp T R. (2013) Identification of a second Nutlin-3 responsive interaction site in the N-terminal domain of MDM2 using hydrogen/deuterium exchange mass spectrometry. Proteomics.
84. Dastidar S G, Lane D P, Verma C S. (2012) Why is F19Ap53 unable to bind MDM2? Simulations suggest crack propagation modulates binding. Cell Cycle 11: 2239-2247.
85. Verkhivker G M (2012) Simulating molecular mechanisms of the MDM2-mediated regulatory interactions: a conformational selection model of the MDM2 lid dynamics. PLoS One 7: e40897.
86. Balius T, Rizzo, R C., (2009) Quantitative prediction of fold resistance for inhibitors of EGFR. Biochemistry 48: 8435-8448.
87. Nair A, Bonin, I., Tossi A, Wels W J, Miertus S. (2002) Computational studies of the resistance patterns of mutant HIV-1 aspartic proteases towards ABT-538 (ritonavir) and design of new derivatives. J Mol Graph Model 21: 171-179.
88. Wang W, Kollman, P A., (2001) Computational study of protein specificity: the molecular basis of HIV-1 protease drug resistance. Proc Natl Acad Sci USA 98:14937-14942.
89. Brown C J, Check C F, Verma C S, Lane D P (2011) Reactivation of p53: from peptides to small molecules. Trends Pharmacol Sci 32: 53-62.
90. Bernal F, Tyler A F, Korsmeyer S J, Walensky L D, Verdine G L (2007) Reactivation of the p53 tumor suppressor pathway by a stapled p53 peptide. J Am Chem Soc 129: 2456-2457.
91. Liu M, Li C, Pazgier M, Mao Y, Lv Y, et al. (2010) D-peptide inhibitors of the p53-MDM2 interaction for targeted molecular therapy of malignant neoplasms. Proc Natl Acad Sci USA 107: 14321-14326.
92. See H Y, Lane D P (2010) A novel unstructured scaffold based on 4EBP1 enables the functional display of a wide range of bioactive peptides. J Mol Biol 404: 819-831.
93. Shangary S, Wang S (2009) Small-molecule inhibitors of the MDM2-p53 protein-protein interaction to reactivate p53 function: a novel approach for cancer therapy. Annu Rev Pharmacol Toxicol 49:223-241.
94. Brennan R C, Federico S, Bradley C, Zhang J, Flores-Otero J, et al. (2011) Targeting the p53 pathway in retinoblastoma with subconjunctival Nutlin-3a. Cancer Res 71: 4205-4213.
95. Secchiero P, Bosco R, Celeghini C, Zauli G (2011) Recent advances in the therapeutic perspectives of Nutlin-3. Curr Pharm Des 17: 569-577.

1. A method of determining resistance of a biological molecule to inhibition of its interaction with a target molecule by an inhibitor of said biological molecule, the method comprising the steps of:
a) co-compartmentalizing a gene encoding said biological molecule with said target molecule, or a gene encoding said biological molecule with a gene encoding said target molecule into an aqueous droplet disposed within a water-in-oil emulsion, and
b) assaying for a complex comprising said biological molecule and said target molecule upon expression of said gene encoding said biological molecule and said gene encoding said target molecule,
wherein detection of said complex in the presence of said inhibitor indicates that said biological molecule is resistant to inhibition of its interaction with said target molecule by said inhibitor, and
wherein non-detection of said complex in the presence of said inhibitor indicates that said biological molecule is not resistant to inhibition of its interaction with said target molecule by said inhibitor.

2. The method according to claim 1, wherein said complex comprises said gene encoding said biological molecule.

3. The method according to claim 1, wherein said emulsion comprises a plurality of said aqueous droplets.

4. The method according to claim 1, wherein each aqueous droplet comprises a single variant of the gene encoding said biological molecule.

5. The method according to claim 1, comprising one or more of the following steps:
step a) comprising co-compartmentalizing said inhibitor with said gene encoding said biological molecule and said target molecule, or with said gene encoding said biological molecule and said gene encoding said target molecule into an aqueous droplet disposed within a water-in-oil emulsion; and/or
step b) comprising assaying for said complex comprising said biological molecule and said target molecule upon expression of said gene encoding said biological molecule and said gene encoding said target molecule after rupturing said aqueous droplet and contacting the contents thereof with said inhibitor; and/or
step b) comprising rupturing the aqueous droplet and contacting the contents thereof with a detectable label capable of binding to said complex; and/or
step b) comprising rupturing the aqueous droplet and contacting the contents thereof with a detectable label selected from the group consisting of a magnetic bead label, an antibody, a radioisotope label, a luminescent label, a fluorescent label, an enzyme label, a colloidal metal label, a colored glass bead label, a colored latex bead label, a carbon black label, and combinations thereof; and/or
step c) comprising amplifying said gene encoding said biological molecule in the complex that has been detected and detecting the amplified gene; and/or
identifying a mutation/(s) in said gene encoding said biological molecule in the complex that has been detected; and/or
identifying a mutation/(s) in said gene encoding said biological molecule in the complex that has been detected by sequence analysis; and/or
analyzing in silico the interaction between said biological molecule and/or said target molecule and/or said inhibitor to determine the mechanism of resistance of said biological molecule to inhibition of its interaction with said target molecule by said inhibitor.

6.-12. (canceled)

13. The method according to claim 1, wherein said inhibitor is present at a concentration capable of inhibiting the interaction of a wild-type form of said biological molecule with said target molecule.

14. The method according to claim 13, wherein the concentration of said inhibitor is selected from the group consisting of at least about 1 μM, at least about 2 μM, at least about 5 μM, at least about 10 μM, at least about 50 μM, and at least about 100 μM.

15.-18. (canceled)

19. The method according to claim 1, wherein said protein is HDM2 ubiquitin ligase.

20. (canceled)

21. (canceled)

22. The method according to claim 1, wherein the target molecule is a p53 tumor suppressor protein.

23. (canceled)

24. The method according to claim 1, wherein the inhibitor is a small organic molecule selected from the group consisting of Nutlin 1, Nutlin 2, Nutlin 3, Nutlin 3A and analogues thereof.

25. (canceled)

26. The method according to claim 5, comprising repeating steps a), b) and c) at least once, at least twice, at least three times, at least four times, at least five times, at least six times, at least seven times, at least eight times, more than 10 times, more than 15 times, or more than 20 times.

27. The method according to claim 1, wherein the method is conducted in vitro.

28.-33. (canceled)

34. A prognostic method for determining the receptiveness of a cancer patient to treatment with an anti-cancer drug capable of inhibiting the interaction of HDM2 ubiquitin ligase with p53 tumor suppressor protein, the method comprising the step of:
comparing a gene encoding said HDM2 ubiquitin ligase derived from a sample of the patient against a plurality of HDM2 ubiquitin ligase genes that have been determined to be resistant to the anti-cancer drug by the method according to the method of claim 1; wherein identification of a match between the gene of the patient to at least one gene in said plurality of HDM2 ubiquitin ligase genes that have been determined to be resistant to the anti-cancer drug indicates that said cancer patient may not be receptive to treatment with said anti-cancer drug.

35. (canceled)

36. The method according to claim 34, wherein the anti-cancer drug is Nutlin 3A.

37.-39. (canceled)

40. The method according to claim 1, for selecting a variant form of a biological molecule that is resistant to inhibition of its interaction with a target molecule by an inhibitor of said biological molecule, comprising the steps of providing a plurality of randomly mutated genes encoding said biological molecule, and determining resistance of said biological molecule to inhibition of its interaction with said target molecule by said inhibitor.

41. The method according to claim 40, wherein said plurality of randomly mutated genes encoding said biological molecule comprises at least 10⁷ randomly mutated genes encoding said biological molecule, at least 10⁸ randomly mutated genes encoding said biological molecule, at least 10⁹ randomly mutated genes encoding said biological molecule, or at least 10¹⁰ randomly mutated genes encoding said biological molecule.

42. (canceled)

43. (canceled)

44. A method of restoring the inhibitory activity of a drug on the interaction of a biological molecule with a target molecule, the method comprising the steps of:
i) identifying a variant of said biological molecule that is resistant to inhibition of its interaction with said target molecule by said drug, using the method according to claim 1; and
ii) modifying said drug to restore its inhibitory activity on the interaction of said biological molecule with said target molecule.

45. The method according to claim 44, wherein step i) comprises determining the mechanism of resistance of said biological molecule to inhibition of its interaction with said target molecule by said drug, and step ii) comprises modifying said drug to overcome said mechanism of resistance.

46. The method according to claim 44, further comprising analyzing the structure of said biological molecule determined to be resistant to inhibition of its interaction with said target molecule by said inhibitor.

47. The method according to claim 46, wherein the structure of said biological molecule determined to be resistant to inhibition of its interaction with said target molecule by said inhibitor is analyzed by nuclear magnetic resonance (NMR) or crystallography.
